SEQUENCE LISTING (1 sequence)

SEQ ID NO: 1 - protein, 32 aa, Artificial Sequence
  Tat-Pro ADAM10 709-729 peptide
YGRKKRRQRRRPKLPPPKPLPGTLKRRRPPQP

(19) United States
(12) Patent Application Publication
(10) Pub. No.: US 2016/0310581 A1
(43) Pub. Date: Oct. 27, 2016

(54) ADAM10 INHIBITION TO TREAT FRAGILE X SYNDROME

(71) Applicants: VIB VZW, Gent (BE); KATHOLIEKE UNIVERSITEIT LEUVEN, K.U.LEUVEN R&D, Leuven (BE)

(72) Inventors: Claudia Bagni, Heverlee (BE); Emanuela Pasciuto, Leuven (BE); Monica Di Luca, Milano (IT); Elena Marcello, Milano (IT); Fabrizio Gardoni, Caponago (IT)

(21) Appl. No.: 15/104,211

(22) PCT Filed: Dec. 11, 2014
(86) PCT No.: PCT/EP2014/077374

§ 371 Date: Jun. 13, 2016

(30) Foreign Application Priority Data
Dec. 11, 2013 (EP) 13196710.1

(51) Int. Cl. A61K 38/48; A61K 38/16; C12N 7/00

(52) U.S. Cl. CPC A61K 38/4886; C12N 7/00; A61K 38/162; C12Y 304/24081; C12N 2740/16322; C12N 2740/16311

(57) ABSTRACT

The present application relates to Fragile X syndrome and the treatment thereof. It was found that ADAM10 (A Dis-integrin And Metalloproteinase domain 10), the most likely candidate for α-secretase, involved in proteolytic cleavage of APP at the plasma membrane, was upregulated in Fmr1 KO mice, a model for Fragile X disease. Moreover, it could be shown that reducing ADAM10 activityin vitro and in vivo, improves the Fragile X phenotype, i.a. by rescuing spine dysmorphogenesis and exaggerated mGluR-dependent LTD.

### FIELD OF THE INVENTION

The present application relates to the Fragile X syndrome (FXS) and the treatment thereof. It was found that ADAM10 (A Disintegrin And Metalloproteinase domain 10), the most likely candidate for α-secretase, involved in proteolytic cleavage of APP at the plasma membrane, was upregulated in the Fmr1 KO mice, a model for FXS. Moreover, it could be shown that reducing ADAM10 activity in vivo, improves the Fragile X phenotype, i.a. by rescuing spine dysmorphogenesis and exaggerated mGluR-dependent LTD—this results both in altered spine morphology and beneficial behavioural effects.

### BACKGROUND

Proper synaptic contacts, trans-synaptic signalling, and structural remodelling are all essential events for synapses formation, maturation and stabilization during brain development (Waites et al., 2005; Yoshihara et al., 2009; Yuste and Bonhoeffer, 2004). Many molecules contribute to these processes, and the fine-tuning of these different proteins is fundamental for synaptic structure, establishment of synaptic networks and finally neuronal plasticity (Gerrow et al., 2006; Sheng and Hoogenraad, 2007). Abnormal spine morphology is associated with various neurodevelopmental diseases such as Down syndrome, Rett syndrome, Autism Spectrum Disorder (ASD) (Betancur et al., 2009; Hutsler and Zhang, 2010) and Fragile X syndrome (FXS) (Fiala et al., 2002; Kaufmann and Moser, 2000; Penzes et al., 2011; Purpura, 1974).

FXS is the most common form of inherited intellectual disability. FXS patients exhibit moderate to severe mental retardation and about 30% of FXS children meet criteria for ASD (Jacquemont et al., 2007; Wang et al., 2010; Bagni et al J Clin Invest 2012). At the cellular level, neurons of FXS patients show an increased number of dendritic spines, which appear long, thin and tortuous (Hinton et al., 1991; Irwin et al., 2001; Rudelli et al., 1985). This dysmorphic spine phenotype is likely causative of the cognitive deficits, behavioural disorders, anxiety and susceptibility to epilepsy observed in patients (Belmonte and Bourgeron, 2006; Jacquemont et al., 2007). Interestingly, the Fragile X mouse model (Fmr1 KO), which exhibits similar morphological alterations, shows a similar behavioral phenotype (Comery et al., 1997; Cruz-Martin et al., 2010; McKinney et al., 2005; Pfeiffer and Huber, 2009).

FXS is due to mutations or absence of the Fragile X Mental Retardation Protein, FMRP, an RNA binding protein highly expressed in brain and testis. Through four RNA-binding domains, FMRP associates with messenger RNAs (mRNAs) encoding pre- and postsynaptic proteins (Darnell et al., 2011), and regulates multiple steps of their metabolism, such as dendritic transport, stability and translation (Bagni and Greenough, 2005; Bassell and Warren, 2008; De Rubeis and Bagni, 2010). Therefore, loss of FMRP compromises the tuned expression of a variety of proteins during development. One of the mechanisms through which FMRP can inhibit the translation of synaptic mRNAs depends on its interaction with the Cytoplasmic FMRP Interacting Protein 1 (CYFIP1), a neuronal eIF4E-binding protein (Napoli et al., 2008; De Rubeis et al., 2013). Noteworthy, among the mRNAs regulated by the FMRP-CYFIP1 complex is the transcript encoding the Amyloid precursor protein (APP) (Westmark and Malter, 2007; Napoli et al., 2008).

APP is a type I transmembrane protein produced in brain microglia, astrocytes, oligodendrocytes, and neurons. APP has a central role in the pathobiology of Alzheimer's Disease (AD) and is also shown to be deregulated in neurodevelopmental disorders like Down Syndrome (Glenner and Wong, 1984) and, more recently, FXS and ASD (Ray et al., 2011; Westmark et al., 2011).

In fact, increasing evidence support a wide range of functions for APP in both developing and adult brain such as neurite outgrowth, synaptogenesis, transmembrane signal transduction, cell adhesion, protein trafficking, calcium metabolism (Hoe et al., 2012; Reinhard et al., 2005; Weyer et al., 2011; Zheng and Koo, 2011). Consistent with its physiological functions, APP expression is high during cortical spine formation and progressively declines after synaptic maturation (Moya et al., 1994). However, APP effects on dendritic spine density are still controversial (Hoe et al., 2012; Jung and Herms, 2011; Lee et al., 2010b). Both increased density (Bittner et al., 2009) and reduction in mature spine density (Lee et al., 2010b) have been reported in the cerebral cortex of APP-deficient mice. Moreover, overexpression of APP increases spine number and promotes peripheral and central synaptogenesis (Lee et al., 2010b; Wang et al., 2009), whereas APP knockdown reduces spine density in hippocampal neurons (Hoe et al., 2012; Jung and Herms, 2011; Lee et al., 2010b).

APP is proteolyzed into various fragments during its intracellular trafficking and the tight coordination of the processing is important for neuronal physiology and pathobiology (Zhang et al., 2011). On the neuronal cell surface, APP undergoes a rapid cleavage by the α-secretase, which generates a secreted form of APP (sAPP α) and a C-terminal fragment (CTF α). sAPP α is upregulated during synaptogenesis, is neurotrophic and neuroprotective (Araki et al., 1991; Copanaki et al., 2010; Milward et al., 1992; Moya et al., 1994). In vivo application of sAPP α increases synaptic density, cortical synaptogenesis and memory retention (Bell et al., 2008; Roch et al., 1994). ADAM10 is the α-secretase required for constitutive sAPP α generation in neurons (Jorissen et al., 2010; Kuhn et al., 2010; Lammich et al., 1999). ADAM10 synaptic localization and activity are important for synaptic morphology (Malinverno et al., 2010) and its overexpression promotes cortical synaptogenesis (Bell et al., 2008). A different APP processing pathway, mediated by the β-secretase BACE1, cleaves APP, mainly in the endosomes, generating a soluble form of APP (sAPPβ) and a C-terminal membrane-associated fragment (CTFβ). This pathway ultimately leads to the formation of β-amyloid (Aβ40-42) (Reinhard et al., 2005; Zhou et al., 2011). Overproduction and accumulation of Aβ in the brain is critical for AD progression, although a modulatory role on neurotransmission and memory formation has been recently proposed (Morley et al., 2010; Plant et al., 2003).

Noteworthy, increased levels of secreted APP and reduced Aβ-40 and Aβ-42 in the plasma have been reported in a large set of autistic children (Ray et al., 2011). Moreover abnormal Aβ levels have been documented in blood and brain of FXS patients (Sokol et al., 2006; Sokol et al., 2011). These recent findings further support a possible dual role of APP in mental retardation and neurodegeneration.

FMRP regulates both basal and activity-induced APP expression (Napoli et al., 2008; Westmark and Malter, 2007). App mRNA has been found in the FMRP-Cyfip1 complex and APP levels are increased in CYFIP1 heterozygous mice (Napoli et al., 2008) while at synapses activation of class I metabotropic glutamate receptors (mGluRs) fails to enhance APP synthesis if FMRP is absent ((Westmark and Malter, 2007). Westmark and colleagues have recently reported a reduction in spine length and mGluR-dependent synaptic depression in Fmr1^{-/-}App^{+/-} neurons (Westmark et al., 2011).

As there is currently no effective cure available for Fragile X syndrome, there is a clear need for therapeutic advances in this field. It would be particularly advantageous if these therapies had a sound molecular basis and are able to overcome the deficits that develop from a very early age.

### SUMMARY

Here we show that, in the absence of FMRP, APP and its metabolite sAPPα accumulate in brain and contribute to the spine malformation observed in the Fragile X Syndrome. Both APP and the α-secretase ADAM10 are excessively expressed in brains from Fmr1 KO mice and the mRNAs encoding for these proteins are detected in the FMRP complex. Importantly, proper spine density and morphology can be restored in Fmr1 KO neurons by modulating APP expression and sAPPα release, e.g. by Adam10 inhibition. This offers new therapeutic opportunities for treating FXS.

Accordingly, it is an object of the invention to provide APP modulating compounds for use in treatment of Fragile X syndrome. Particularly, it is envisaged to provide sAPPα modulating compounds for use in treatment of Fragile X syndrome. Most particularly, it is envisaged to provide inhibitors of Adam10 for use in treatment of Fragile X syndrome.

This is equivalent as saying that methods of treating Fragile X syndrome in a subject in need thereof are provided, comprising administering APP modulating compounds to the subject. Particularly, methods of treating Fragile X syndrome in a subject in need thereof are provided, comprising administering sAPPα modulating compounds to the subject. Most particularly, methods of treating Fragile X syndrome in a subject in need thereof are provided, comprising administering inhibitors of Adam10 to the subject.

Different inhibitors of Adam10 are known in the art. Moreover, novel inhibitors (e.g. siRNA, antibodies, nanobodies, small molecules) can be generated using methods known in the art, or using conventional screening assays. According to particular embodiments, the Adam10 inhibitor is selected from an anti-ADAM10 peptide (e.g. Marcello et al., 2007), G1254023X (e.g. Hoettecke et al., 2010) and triptolide (e.g. Soundararajan et al., 2010). According to even further particular embodiments, the anti-ADAM10 peptide contains the sequence YGRKKRRQRRRPKLPPPKPLPGTLKRRRPPQP (SEQ ID NO:1). According to yet even further particular embodiments, the anti-ADAM10 peptide is the Tat-Pro ADAM10⁷⁰⁹⁻⁷²⁹ peptide, i.e. consists of the sequence YGRKKRRQRRRPKLPPPKPLPGTLKRRRPPQP (SEQ ID NO:1). This cell-permeable peptide is obtained by linking the 11 aa human immunodeficiency virus Tat transporter sequence to the 21 aa sequence (Aarts et al., 2002) corresponding to ADAM10 proline rich domains (Marcello et al., 2007). The peptide crosses the blood-brain barrier and penetrates neurons (Marcello et al., 2007).

It is particularly envisaged that administration of the inhibitor (or the use of the inhibitor in treatment) will result in (at least partial) rescue of spine dysmorphogenesis and possibly some of the other impaired pathways (protein synthesis and excessive mGluR activity). Thus, according to particular embodiments, the treatment of Fragile X syndrome is (at least in part) rescue of the spine malformations observed in Fragile X syndrome. It is the cellular readout used so far for all the molecules tested for FXS ameliorations (see Osterweil E K et al Neuron 2013, Michalon A et al Neuron 2012).

### BRIEF DESCRIPTION OF THE FIGURES

FIG. 1. APP is highly expressed in FXS.

(A) APP levels are increased in FXS human lymphoblastoid cells. Left panel, Western blot analysis of APP and FMRP levels in cerebellum from healthy controls (lane 1-6) and FXS (lane 7-12). Middle panel, the histogram shows the quantification of APP normalized for Vinculin. Error bars represent SEM; *P<0.05, Student's t test. (B) APP levels in the frontal cortex of Fragile X patients. Left panel, Western blot analysis of APP levels in cortex from healthy controls (lane 1-3) and FXS (lane 4-7). Middle panel, the histogram shows the quantification of APP normalized for Vinculin. Error bars represent SEM; *P<0.05, Student's t test. Rigth panel, App mRNA levels are comparable in controls and FXS. Error bars represent SEM. (C) APP levels in the cerebellum of Fragile X patients. Left panel, Western blot analysis of APP and FMRP levels in cerebellum from healthy controls (lane 1-8) and FXS (lane 9-12). Middle panel, the histogram shows the quantification of APP normalized for Vinculin. Error bars represent SEM; **P<0.05, Student's t test. Right panel, App mRNA levels are comparable in controls and FXS. Error bars represent SEM. (D) In Fmr1 KO APP levels are upregulated during brain development. Wild-type (WT) and Fmr1 KO brain were analyzed during development by Western blot for APP, aCaMKII, FMRP, GAPDH and Vinculin. Left, protein analysis from P7, P14, P21, P30 and P90. Right, histograms show quantified protein levels normalized for GAPDH. This ratio was set to 1 in WT mice. Error bars represent standard error of the mean (SEM); *P<0.05; **P<0.01, Student's t test, n=5.

FIG. 2. FMRP regulates App mRNA translation

(A) APP levels are upregulated in the cortex of Fmr1 KO mice. Left panel, Western blot analysis of APP levels in cortex from WT (lane 1) and Fmr1 KO (lane 2) mice at P21. Right panel, the histogram shows the quantification of APP normalized for GAPDH. This ratio was set to 1 in WT mice. Error bars represent SEM; **P<0.01, Student's t test, n=3. (B) App mRNA is specifically associated with FMRP. FMRP was immunoprecipitated and the associated mRNAs revealed by RT-PCR. Specificity of the complex is shown by the presence of a well-known FMRP target mRNA (αCamKII) and absence of a non-target mRNA (Cyp46). Lane 1 (Input 1/20), lane 2 (aFMRP), lane 3 (CTRL IgGs). (C) App, αCaMKII and βactin mRNA basal levels are comparable in WT and Fmr1 KO cortices. RT-qPCR was performed from mouse cortex and App levels were normalized to Cyp46 mRNA. Error bars represent SEM, n=4. (D) App mRNA is differentially translated in Fmr1 KO cortices. Cortical cytoplasmic extracts were fractionated along a 10-60% sucrose gradient. Polysome-mRNP distribution was determined collecting twelve fractions while reading the absorbance at 254 nm. For each fraction, the RT-qPCR quantification of App mRNA was normalized to βactin mRNA. Shown is a representative polysomal-mRNP profile. Error bars represent SEM, *P<0.05 **P<0.01, Student's t test, n=4. (E) App mRNA dissociates from FMRP upon mGluR stimulation. FMRP was immunoprecipitated from cortical neurons following 5 min stimulation with DHPG (100 μM). The amount of associated App mRNA in control (CTRL, not stimulated) and stimulated (DHPG) cells was quantified by RT-qPCR. Immunoprecipitated App mRNA was normalized to the input and the value of App mRNA associated to the IgGs IP considered as background. Histone 3.3 mRNA was used as negative control. Error bars represent SEM, ***P<0.001, Student's t test, n=4. (F) APP expression is regulated by mGluR activatio. Left panel, Western blot analysis of APP and αCamKII (positive control) levels in control (CTRL) and stimulated (DHPG) neurons at DIV 14. Right panel, the histogram shows the quantification of APP and αCamKII normalized for Vinculin. This ratio was set to 1 in control. Error bars represent SEM; **P<0.01, Student's t test, n=4.

FIG. 3. Processing of APP is impaired in Fmr1 KO mice and leads to an enhanced sAPPa release.

(A) Cell-surface APP is reduced in Fmr1 KO cortical neurons. WT and Fmr1 KO cortical neurons were stained for total APP (left panels) or surface APP (right panels) Shown are representative dendritic fragments (>50 μm distance from cell body). Scale bar, 5 μm. Histograms show quantified total and surface protein levels as mean fluorescence intensity (M.F.I). Error bars represent standard error of the mean (SEM); ***P<0.001, Student's t test. (B) Surface proteins from WT and Fmr1 KO cortical neurons were biotinylated, captured with streptavidin-dynabeads and analyzed for APP levels by Western blot. Left panel: representative Western blot. Lanes 1 and 2, total input (1/10), lanes 3 and 4, pulled-down surface APP normalized to the input. GAPDH was used for normalization while NR2A to monitor membrane proteins. Right panel, histograms show the quantification. Changes of total input versus surface APP are expressed as percentage to WT. Error bars represent SEM; **P<0.01, One Sample t test, n=4. (C) Soluble APPa is increased in cortical neurons. sAPP level was analyzed by Western blot in conditioned medium from WT and KO cortical neurons. Left, protein analysis of sAPP and Coomassie staining of loaded proteins. Normalized APP levels over Coomassie were set to 1 in WT mice. Error bars represent SEM; *P<0.05; Student's t test, n=4. (D) Products of the APP processing are dysregulated in P21 Fmr1 KO brains. Left panel, scheme representing APP products following α and β secretase cleavage. Central panel, representative Western blot with the levels of sAPP, sAPPa and sAPPβ in the soluble fraction (FIG. 8, 11). Left panel, the histograms show the quantification of the three products over Coomassie. Error bars represent SEM; *P<0.05, One Sample t test, n=4. (E) A β levels are reduced in Fmr1 KO brains. ELISA assay was performed to measure A β40 generation in WT and Fmr1 KO cortices. The amount of A β was normalized to brain weight and expressed as percentage of wild-type. Error bars represent SEM. *P<0.05, Student's t test, n=4. (F) APP level is increased in synaptoneurosomes of Fmr1 KO mice. Western blots of WT and Fmr1 KO synaptoneurosomes. Right panel shows the quantification of APP expression normalized to GAPDH. This ratio in WT mice was set at 1. Error bars represent SEM; ***P<0.001, Student's t test, n=4. (G) Generation of α and β secretase products is affected in Fmr1 KO synaptic compartments. Synaptic membranes from WT and Fmr1 KO mice were analysed for APP, APP CTFs, FMRP and Vinculin levels by Western blot. The histograms show quantified proteins after normalization to Vinculin, expressed as percentage of WT. Error bars represent SEM; *P<0.05, **P<0.01, One sample t test, n=6.

FIG. 4. FMRP modulates the level of the APP processing enzyme ADAM10.

(A) ADAM10 is highly expressed in Fmr1 KO. Left panel, representative Western blot showing ADAM10 expression level in WT (lane 1) and Fmr1 KO (lane 2) cortex. Black arrowheads: mature proteins; white arrowheads: immature proteins. Right panel, histograms representing the quantification of ADAM10 over GAPDH levels expressed as percentage of WT. Error bars represent SEM; *P<0.05, Student's t test, n=3. (B) Immunofluorescence of ADAM10 in DIV14 cortical neurons. Shown are representative dendritic fragments (>50 μm from cell body). White arrowheads point to spines. Scale bar, 5 μm. (C) Adam10 mRNA is associated with FMRP. First panel: Western blot analysis of immunoprecipitated FMRP. Lane 1: input (1/20), lane 2: aFMRP, lane 3: CTRL IgGs. Second to sixth panels: RT-PCR of FMRP associated mRNAs. Dopamin Receptor D2 mRNA (D₂DR) was used as negative control, αCaMKII mRNA was used as positive control. (D) Adam10 mRNA basal levels are comparable in WT and Fmr1 KO cortices. RT-qPCR was performed from mouse cortex and Adam10 levels were normalized to Cyp46 mRNA. Error bars represent SEM, n=4. (E) Polysome-mRNPs distribution from mouse cortical cytoplasmic extracts. Cytoplasmic extracts were fractionated along a 10-60% sucrose gradient. Twelve fractions were collected while reading the absorbance at 254 nm. Amount of Adam10 and βactin mRNAs in each fraction was quantified by RT-qPCR. Shown is a representative polysomal-mRNP profile. Each fraction shows the quantification of Adam10 mRNA normalized to βactin mRNA in WT and Fmr1 KO. Error bars represent SEM. *P<0.05, **P<0.01, Student's t test, n=3. (F) Surface ADAM10 is increased in Fmr1 KO cortical neurons. Proteins were biotinylated (lanes 1, 2) and captured with streptavidin-dynabeads (pull-down, lanes 3, 4). ADAM10 levels were analyzed by Western blot. GAPDH was used as loading control. (G) ADAM10 expression is increased in synaptoneurosomes from Fmr1 KO mice. Left panel, representative Western blot showing ADAM10, ADAM9, ADAM17 expression level in WT (lane 1) and Fmr1 KO (lane 2). Black arrowheads: mature proteins; white arrowheads: immature proteins. Right panel, histograms representing the quantification of ADAM10 over GAPDH levels expressed as percentage of WT. Error bars represent SEM; **P<0.01, Student's t test, n=4. (H) Cleavage of N-cadherin and Notch is not affected in Fmr1 KO. Left panel, representative Western blot showing N-cadherin full length and C-terminal fragment; middle panel, western blot showing Notch cleavage products S1 (black arrowhead) and S2 (black arrowhead) in WT (lane 1) and Fmr1 KO (lane 2) cortex. Right panel, histograms representing protein quantification over a-tubulin levels expressed as percentage of WT. Error bars represent SE. n=6.

FIG. 5. sAPPa levels are crucial for spine morphology in FXS.

(I) FXS spine morphology is rescued by APP reduction. Cultured cortical neurons (DIV8) were transfected with lentiviral vectors expressing EGFP-App shRNA or EGFP-CTRL shRNA and analyzed at DIV14. Representative dendritic segments are shown. Top panel, WT neurons transfected with control shRNA. 2^{nd} and 3^{rd} panels, Fmr1 KO neurons transfected with control shRNA or App shRNA, respectively. 4^{th} panel, Fmr1 KO neurons transfected with App shRNA, and treated with sAPPa. Scale bar, 5 μm. (II) Spine analysis was performed according to the scheme. Quantification of the spine density (III) and spine length (IV). Histograms represent the mean values. (V) Spine density of the different spine types. Error bars represent SEM, *P<0.05, **P<0.01, ***P<0.001, n.s, non-significant, one-way ANOVA, followed by Student's t test and Bonferroni correction. n=10 neurons. For each condition 350-500 spines were analyzed. (VI) Ratio between mature and immature spines. ***P<0.001, Chi-squared test, Bonferroni correction.

FIG. 6: Modulation of ADAM10 reduces excessive APP cleavage in the Fmr1 KO and ameliorates spine dysmorphogenesis.

(A) The tissue inhibitor of metalloproteinase-1 (TIMP-1) reduces sAPP release in neurons. WT and Fmr1 KO cortical neurons were treated with 15 nM TIMP-1 for 4 h to inhibit ADAM10 activity. Media was collected and sAPP levels measured by western blot (Left panel). Right panel, histogram representing the quantification of sAPP over comassie staining. Error bars represent SEM; *P<0.05, Student's t test, n=3. (B) Treatment with TAT-Pro reduces sAPP release in neurons DIV15 cortical neurons were treated with TAT-Ala and TAT-Pro ADAM10 peptides (10 uM, 1 h). Media was collected and sAPP levels measured by western blot (Left panel). Right panel, histogram representing the quantification of sAPP over comassie staining. Error bars represent SEM; *P<0.05, Student's t test, n=3 (C) ADAM10 activity can be modulated in vivo in the Fmr1 KO. Young mice (P21) received a single intraperitoneal injection of either TAT-Pro (3 nmol/g) or TAT-Ala peptide (3 nmol/g). The effects on sAPPα relase in WT and KO brain were monitored after 24 h by western blot. Right panel, histogram representing the quantification of sAPPa over comassie staining. Error bars represent SEM; *P<0.05, Student's t test, n=5 (D) TAT-Ala and TAT-Pro ADAM10 peptides were injected in vivo (3 nmol/g, 24 h; 14-d-old mice) in WT and Fmr1 KO mice. Morphological analysis of dendritic spines was conducted using a diolistic gene-gun system to propel Dil-coated particles into hippocampal sections of fixed brain tissue of TAT-Pro- and TAT-Ala treated mice. E, Histogram of average spine length, head width abd density *P<0.05, TAT-Ala WT vs TAT-Ala KO; #P<0.05, TAT-Pro KO vs TAT-Ala KO

FIG. 7. FMRP modulates APP metabolism at synapses: a working model

(A) FMRP regulates APP and Adam10 mRNAs and consequently their protein levels in the cortex (1). Newly synthesized APP molecules mature through the constitutive secretory pathway (2) (SV, Secretory vesicles). At the cell surface APP can be cleaved by αsecretase (ADAM10) and sAPPα is released, non-processed APP is rapidly internalized in early endosomes (EE). APP can shuttle from early endosomes back to the Golgi (3) or alternatively can transit through late endosomes (LE), where β-cleavage (BACE1) occurs (4). (B) In absence of FMRP, APP and ADAM10 are both upregulated leading to sAPPα accumulation and preventing Aβ generation. Excess of sAPPα affects spine morphology and contributes to the immature phenotype observed in the Fmr1 KO mouse and possibly FXS patients.

FIG. 8. APP regions recognized by the antibodies used in this study.

Shown are also the cleavage sites by α, β, γ secretase activities.

FIG. 9. App mRNA is associated with FMRP.

(A) Association of App mRNA with FMRP complex. FMRP was immunoprecipitated (ocFMRP) from total brain lysates, and associated mRNAs were detected by RT-PCR. Neuronal D2DR and αCaMKII mRNAs were used as negative and positive controls, respectively. Lanes 1-2: Inputs (1/20); lanes 3-4: αFMRP from WT and Fmr1 KO. (B) Quantification of App mRNA levels in total brain. RT-qPCR was performed from total mouse brain to assess the steady state levels of App mRNA after normalization to D2DR mRNA. Error bars represent SEM, n=4.

FIG. 10. αCaMKII is more efficiently translated in Fmr1 KO brains.

Polysome-mRNP distribution of αCaMKII and βactin mRNAs. Cytoplasmic extracts were fractionated along a 10%-60% sucrose gradient. Twelve fractions were collected while reading the absorbance at 254 nm. Fractions 1-7 (polysomes) and 8-12 (mRNPs) were pooled and the mRNA concentration in each pool was quantified by RT-qPCR (see Experimental Procedures). Histograms represent the translational efficiency—as expressed by the polysome over mRNP ratio—of αCaMKII and βactin mRNAs in WT and Fmr1 KO. Error bars represent standard error of the mean. *P<0.05, Student's t test, n=3.

FIG. 11. Full length APP and sAPP can be detected in different biochemical brain fractions.

Left: scheme of brain fractionation. The S1 fraction contains soluble APP (lane 1), while S2 (lane 2) and P1 fractions (lane 3) contain the proteins present in membranes and lipid rafts, respectively. Right panel: forebrains were fractionated, and APP was detected using two different antibodies to discriminate full length APP (A8717) and soluble APP (22C11).

FIG. 12. APP is present in synaptoneurosomes and synaptic Detergent Resistent Microdomains (DRM).

APP, FMRP, GAPDH, Vinculin, Syntaxin 1A, Synaptophysin, NR2A, PSD95 and Flotillin 1, were detected in synaptoneurosomes (lane 1) and DRMs (lane 2) by Western blot analysis.

FIG. 13. Validation of the APP silencing.

(A) Mouse Embryonic Fibroblasts (MEFs) were transfected with EGFP-App shRNA or EGFP-CTRL shRNA vectors. Cells were stained for APP (A8717 antibody) after 72 hrs. Scale bar corresponds to 20 μm. (B) DIV8 cortical neurons were transfected with EGFP-App shRNA vector. At DIV14 cells were stained for APP (A8717 antibody). Scale bar corresponds to 10 μm. (C) Representative Western blot showing APP expression during in vitro development of WT and Fmr1 KO cortical neurons. The bottom left panel shows the quantification of APP levels normalized to βactin; bottom right panel represents the quantification of APP expressed as percentage of the variation between WT and KO. Error bars represent SEM; *P<0.05, **P<0.01, Student's t test, n=3.

FIG. 14. APP and sAPP a contribute to the spine defects of Fmr1 KO neurons.

Distribution of the spines along four morphological classes, namely mushroom (black), stubby (dark gray), long thin (light gray) and filopodia (white) in the following conditions: WT and KO CTRL shRNA; KO App shRNA; KO App shRNA+sAPP α. Chi-squared test, *P<0.05, ***P<0.001.

FIG. 15. Modulation of ADAM10 activity reduces excessive sAPPα and ameliorates Long Term Depression (LTD) and behavior in the Fmr1 KO mice.

(A) Fmr1 KO mice exhibit enhanced mGluR-LTD. n=15 slices from 9 mice/genotype (B) TAT-Ala or Tat-Pro peptide does not impact LTD in the WT mice, whereas (C) Tat-Pro prevents mGluR-LTD in the Fmr1 KO mice (F=20, 75), p<0.0002 (TAT-Pro n=9 slices from 6 mice, TAT-Ala n=8 slices from 4 mice). Solid bars indicate the duration of the bath application of DHPG (30 μM, 15 min). Representative traces (right panels) showing fEPSP before (1), 10 min after (2) and 240 min after (3) DHPG application. The stimulus artefact is blanked to ease interpretation. Data are shown as the means±SEM. (D) Effect of the Tat-Pro peptide treatment on the performance of WT and Fmr1 KO mice in the open field. Histograms represent the distance and the speed of the animals in the open field arena. One way Anova followed by Dunnett's multiple comparisons test. *p<0.05. (E) Effect of the peptide on the working memory in the T-maze. Left panel, table showing the preference index for the novel arm in test 2 (1, no preference; >1, preference for the Novel Arm; <1 preference for Familiar Arm. Right panel, histograms show the preference for the novel arm in the test 2 of the T-maze test. (n=9 WT; n=9 Fmr1 KO; n=9 TAT-Pro WT; n=12 Tat-Pro Fmr1 KO).

### DETAILED DESCRIPTION

Definitions

The present invention will be described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 2^{nd} ed., Cold Spring Harbor Press, Plainsview, N.Y. (1989); and Ausubel et al., Current Protocols in Molecular Biology (Supplement 47), John Wiley & Sons, New York (1999), for definitions and terms of the art. The definitions provided herein should not be construed to have a scope less than understood by a person of ordinary skill in the art.

The term “ADAM10” as used herein refers to the ADAM metallopeptidase domain 10 gene (Gene ID: 102 for the human gene, also indicated with the Hugo Gene Nomenclature Committee (HGNC) number: 188), also known as kuz, AD10, MADM, CD156c, or HsT18717, and its products. Members of the ADAM family are cell surface proteins with a unique structure possessing both potential adhesion and protease domains. This gene encodes an ADAM family member that cleaves many proteins including TNF-alpha, E-cadherin and APP. The “ADAM10 gene product” as used herein typically refers to what is transcribed or translated from the ADAM10 gene, such as ADAM10 mRNA and ADAM10 protein. Different isoforms or variants of Adam10 mRNA and the resulting ADAM10 protein isoforms or variants are envisaged within the term ADAM10 gene product. Fragments of a ADAM10 gene product are also envisaged, as long as they are functionally active.

An “ADAM10 inhibitor” as used herein refers to a substance that can interfere with the function of the ADAM10 gene product, either at the DNA level (by inhibiting the formation of ADAM10 gene product, i.e. by preventing or interfering with transcription), at the RNA level (by neutralizing or destabilizing mRNA to prevent or interfere with translation) or at the protein level (by neutralizing or inhibiting ADAM10 protein). Typically, the ultimate functional effect of an ADAM10 inhibitor will be inhibition of the cleavage activity of ADAM10, although this can be achieved indirectly (e.g. at the DNA level).

The term “Fragile X syndrome” as used herein refers to a genetic syndrome (OMIM entry #300624) characterized by the expansion of the CGG trinucleotide repeat affecting the Fragile X mental retardation 1 (FMR1) gene on the X chromosome, resulting in a failure to express the fragile X mental retardation protein (FMRP), which is required for normal neural development. This results i.a. in a spectrum of intellectual disabilities.

The Fragile X mental retardation protein (FMRP) is an RNA-binding protein that regulates key aspects of neuronal RNA metabolism and its absence or mutations lead to the Fragile X Syndrome (FXS). Alterations in spine density and morphology are FXS micro-anatomical hallmarks. Among the transcripts regulated by FMRP there is the mRNA encoding the amyloid precursor protein (APP), a protein critical for Alzheimer's Disease (AD) pathogenesis but also recently shown to play a role in synaptic contacts formation during development. APP upregulation in FXS (Westmark and Malter 2007) and its cleavage deregulation in ASD (Ray et al., 2011) prompted us to investigate how such a deregulation affects spine morphology in FXS.

Here we show that, in the absence of FMRP, APP and its metabolite sAPPα accumulate in brain and contribute to the spine malformation observed in the Fragile X Syndrome. Both APP and the α-secretase ADAM10 are excessively expressed in brains from Fmr1 KO mice and the mRNAs encoding for these proteins are detected in the FMRP complex. APP expression is excessive in Fmr1 KO animals during development and in adulthood due to exaggerated translation. Despite an increase of APP synthesis, FMRP loss significantly decreases APP surface expression due to impairment in the cleavage pathway. Aβ generation is reduced in young Fmr1 KO mice while α-cleavage is enhanced due to an increased ADAM10 activity. The unbalanced α-cleavage is due to the excessive expression of ADAM10, whose translation might also be controlled by FMRP. We also found that this deregulated pathway can contribute to the spine dysgenesis observed in FXS, since proper spine density and morphology can be restored in Fmr1 KO neurons by modulating APP expression and sAPPα release. Indeed, a global reduction of APP levels in Fmr1 KO neurons restores normal spine morphology while sAPPα administration prevented this effect, thus indicating that alterations in APP and in its non-amyloidogenic cleavage contribute to spine defects in FXS. These findings show that FMRP regulates key molecules involved in APP processing linking two different diseases such as mental retardation and neurodegeneration. Importantly, the APP—ADAM10 pathway has not been implicated in Fragile X before, and this offers new therapeutic opportunities for treating FXS.

Accordingly, it is an object of the invention to provide APP modulating compounds for use in treatment of Fragile X syndrome. Particularly, it is envisaged to provide sAPPα modulating compounds for use in treatment of Fragile X syndrome. Preferably, the sAPPα modulating compounds are compounds that reduce the levels of sAPPα. Most particularly, it is envisaged to provide inhibitors of Adam10 (also upregulated in FXS) for use in treatment of Fragile X syndrome.

This is equivalent as saying that methods of treating Fragile X syndrome in a subject in need thereof are provided, comprising administering APP modulating compounds to the subject. Particularly, methods of treating Fragile X syndrome in a subject in need thereof are provided, comprising administering sAPPα modulating compounds to the subject. Most particularly, methods of treating Fragile X syndrome in a subject in need thereof are provided, comprising administering inhibitors of Adam10 to the subject.

A subject typically is a vertebrate subject, more typically a mammalian subject, most typically a human subject.

Inhibition of Adam10 can be achieved at four levels. First, at the DNA level, e.g. by removing or disrupting the ADAM10 gene, or preventing transcription to take place (in both instances preventing, or partially preventing (which is explicitly envisaged), synthesis of the ADAM10 gene product). Second, at the RNA level, e.g. by preventing efficient translation to take place—this can be through destabilization of the mRNA so that it is degraded before translation occurs from the transcript, or by hybridizing to the mRNA. Third, at the protein level, e.g. by binding to the protein, inhibiting its function, and/or marking the protein for degradation. Fourth, in case the effective protein product has to be translocated (i.e. at synapse) to be active, inhibiting its transport.

If inhibition is to be achieved at the DNA level, this may be done using gene therapy to knock-out or disrupt the ADAM10 gene. As used herein, a “knock-out” can be a gene knockdown or the gene can be knocked out by a mutation such as, a point mutation, an insertion, a deletion, a frameshift, or a missense mutation by techniques known in the art, including, but not limited to, retroviral gene transfer. Another way in which genes can be knocked out is by the use of zinc finger nucleases. Zinc-finger nucleases (ZFNs) are artificial restriction enzymes generated by fusing a zinc finger DNA-binding domain to a DNA-cleavage domain. Zinc finger domains can be engineered to target desired DNA sequences, which enables zinc-finger nucleases to target unique sequence within a complex genome. By taking advantage of endogenous DNA repair machinery, these reagents can be used to precisely alter the genomes of higher organisms.

The knock-out of the ADAM10 gene may be limited to the tissue where Adam10 expression is problematic, e.g. the brain. Apart from tissue-specific inhibition of ADAM10 gene product function, the inhibition may also be temporary (or temporally regulated). It is particularly envisaged that ADAM10 is inhibited in young subjects, but not in adults. This both because Fragile X is a neurodevelopmental disease, and to avoid unnecessary interference with APP processing in aging subjects.

Temporally and tissue-specific gene inactivation may for instance also be achieved through the creation of transgenic organisms expressing antisense RNA, or by administering antisense RNA to the subject. An antisense construct can be delivered, for example, as an expression plasmid, which, when transcribed in the cell, produces RNA that is complementary to at least a unique portion of the cellular Adam10 mRNA.

A more rapid method for the inhibition of gene expression is based on the use of shorter antisense oligomers consisting of DNA, or other synthetic structural types such as phosphorothiates, 2′-O-alkylribonucleotide chimeras, locked nucleic acid (LNA), peptide nucleic acid (PNA), or morpholinos. With the exception of RNA oligomers, PNAs and morpholinos, all other antisense oligomers act in eukaryotic cells through the mechanism of RNase H-mediated target cleavage. PNAs and morpholinos bind complementary DNA and RNA targets with high affinity and specificity, and thus act through a simple steric blockade of the RNA translational machinery, and appear to be completely resistant to nuclease attack. An “antisense oligomer” refers to an antisense molecule or anti-gene agent that comprises an oligomer of at least about 10 nucleotides in length. In embodiments an antisense oligomer comprises at least 15, 18 20, 25, 30, 35, 40, or 50 nucleotides. Antisense approaches involve the design of oligonucleotides (either DNA or RNA, or derivatives thereof) that are complementary to an mRNA encoded by polynucleotide sequences of ADAM10. Antisense RNA may be introduced into a cell to inhibit translation of a complementary mRNA by base pairing to it and physically obstructing the translation machinery. This effect is therefore stoichiometric. Absolute complementarity, although preferred, is not required. A sequence “complementary” to a portion of an RNA, as referred to herein, means a sequence having sufficient complementarity to be able to hybridize with the RNA, forming a stable duplex; in the case of double stranded antisense polynucleotide sequences, a single strand of the duplex DNA may thus be tested, or triplex formation may be assayed. The ability to hybridize will depend on both the degree of complementarity and the length of the antisense polynucleotide sequence. Generally, the longer the hybridizing polynucleotide sequence, the more base mismatches with an RNA it may contain and still form a stable duplex (or triplex, as the case may be). One skilled in the art can ascertain a tolerable degree of mismatch by use of standard procedures to determine the melting point of the hybridized complex. Oligomers that are complementary to the 5′ end of the message, e.g., the 5′ untranslated region (UTR) up to and including the AUG translation initiation codon, should work most efficiently at inhibiting translation. However, sequences complementary to the 3′ UTR of mRNAs have recently been shown to be effective at inhibiting translation of mRNAs as well (Wagner, R. (1994) Nature 372, 333 335). Therefore, oligomers complementary to either the 5′, 3′ UTRs, or non-coding regions of a ADAM10 gene could be used in an antisense approach to inhibit translation of said endogenous mRNA encoded by ADAM10 polynucleotides. Oligomers complementary to the 5′ UTR of said mRNA should include the complement of the AUG start codon. Antisense oligomers complementary to mRNA coding regions are less efficient inhibitors of translation but could be used in accordance with the invention. Whether designed to hybridize to the 5′,3′ or non coding region of a said mRNA, antisense oligomers should be at least 10 nucleotides in length, and are preferably oligomers ranging from 15 to about 50 nucleotides in length. In certain embodiments, the oligomer is at least 15 nucleotides, at least 18 nucleotides, at least 20 nucleotides, at least 25 nucleotides, at least 30 nucleotides, at least 35 nucleotides, at least 40 nucleotides, or at least 50 nucleotides in length. A related method uses ribozymes instead of antisense RNA. Ribozymes are catalytic RNA molecules with enzyme-like cleavage properties that can be designed to target specific RNA sequences. Successful target gene inactivation, including temporally and tissue-specific gene inactivation, using ribozymes has been reported in mouse, zebrafish and fruitflies. RNA interference (RNAi) is a form of post-transcriptional gene silencing. The phenomenon of RNA interference was first observed and described in Caenorhabditis elegans where exogenous double-stranded RNA (dsRNA) was shown to specifically and potently disrupt the activity of genes containing homologous sequences through a mechanism that induces rapid degradation of the target RNA. Several reports describe the same catalytic phenomenon in other organisms, including experiments demonstrating spatial and/or temporal control of gene inactivation, including plant (Arabidopsis thaliana), protozoan (Trypanosoma bruceii), invertebrate (Drosophila melanogaster), and vertebrate species (Danio rerio and Xenopus laevis). The mediators of sequence-specific messenger RNA degradation are small interfering RNAs (siRNAs) generated by ribonuclease III cleavage from longer dsRNAs. Generally, the length of siRNAs is between 20 25 nucleotides (Elbashir et al. (2001) Nature 411, 494 498). The siRNA typically comprise a sense RNA strand and a complementary antisense RNA strand annealed together by standard Watson Crick base pairing interactions (hereinafter “base paired”). The sense strand comprises a nucleic acid sequence that is identical to a target sequence contained within the target mRNA. The sense and antisense strands of the present siRNA can comprise two complementary, single stranded RNA molecules or can comprise a single molecule in which two complementary portions are base paired and are covalently linked by a single stranded “hairpin” area (often referred to as shRNA). The term “isolated” means altered or removed from the natural state through human intervention. For example, an siRNA naturally present in a living animal is not “isolated,” but a synthetic siRNA, or an siRNA partially or completely separated from the coexisting materials of its natural state is “isolated.” An isolated siRNA can exist in substantially purified form, or can exist in a non native environment such as, for example, a cell into which the siRNA has been delivered.

The siRNAs of the invention can comprise partially purified RNA, substantially pure RNA, synthetic RNA, or recombinantly produced RNA, as well as altered RNA that differs from naturally occurring RNA by the addition, deletion, substitution and/or alteration of one or more nucleotides. Such alterations can include addition of non nucleotide material, such as to the end(s) of the siRNA or to one or more internal nucleotides of the siRNA, including modifications that make the siRNA resistant to nuclease digestion.

One or both strands of the siRNA of the invention can also comprise a 3′ overhang. A “3′ overhang” refers to at least one unpaired nucleotide extending from the 3′ end of an RNA strand. Thus, in one embodiment, the siRNA of the invention comprises at least one 3′ overhang of from one to about six nucleotides (which includes ribonucleotides or deoxynucleotides) in length, preferably from one to about five nucleotides in length, more preferably from one to about four nucleotides in length, and particularly preferably from about one to about four nucleotides in length.

In the embodiment in which both strands of the siRNA molecule comprise a 3′ overhang, the length of the overhangs can be the same or different for each strand. In a most preferred embodiment, the 3′ overhang is present on both strands of the siRNA, and is two nucleotides in length. In order to enhance the stability of the present siRNAs, the 3′ overhangs can also be stabilized against degradation. In one embodiment, the overhangs are stabilized by including purine nucleotides, such as adenosine or guanosine nucleotides.

Alternatively, substitution of pyrimidine nucleotides by modified analogues, e.g., substitution of uridine nucleotides in the 3′ overhangs with 2′ deoxythymidine, is tolerated and does not affect the efficiency of RNAi degradation. In particular, the absence of a 2′ hydroxyl in the 2′ deoxythymidine significantly enhances the nuclease resistance of the 3′ overhang in tissue culture medium.

The siRNAs of the invention can be targeted to any stretch of approximately 19 to 25 contiguous nucleotides in any of the target ADAM10 mRNA sequences (the “target sequence”), of which examples are given in the application. Techniques for selecting target sequences for siRNA are well known in the art. Thus, the sense strand of the present siRNA comprises a nucleotide sequence identical to any contiguous stretch of about 19 to about 25 nucleotides in the target mRNA.

The siRNAs of the invention can be obtained using a number of techniques known to those of skill in the art. For example, the siRNAs can be chemically synthesized or recombinantly produced using methods known in the art. Preferably, the siRNA of the invention are chemically synthesized using appropriately protected ribonucleoside phosphoramidites and a conventional DNA/RNA synthesizer. The siRNA can be synthesized as two separate, complementary RNA molecules, or as a single RNA molecule with two complementary regions. Commercial suppliers of synthetic RNA molecules or synthesis reagents include Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA) and Cruachem (Glasgow, UK).

Alternatively, siRNA can also be expressed from recombinant circular or linear DNA plasmids using any suitable promoter. Suitable promoters for expressing siRNA of the invention from a plasmid include, for example, the U6 or H1 RNA pol Ill promoter sequences and the cytomegalovirus promoter. Selection of other suitable promoters is within the skill in the art. The recombinant plasmids of the invention can also comprise inducible or regulatable promoters for expression of the siRNA in a particular tissue or in a particular intracellular environment. The siRNA expressed from recombinant plasmids can either be isolated from cultured cell expression systems by standard techniques, or can be expressed intracellularly, e.g. in breast tissue or in neurons.

The siRNAs of the invention can also be expressed intracellularly from recombinant viral vectors. The recombinant viral vectors comprise sequences encoding the siRNAs of the invention and any suitable promoter for expressing the siRNA sequences. Suitable promoters include, for example, the U6 or H1 RNA pol III promoter sequences and the cytomegalovirus promoter. Selection of other suitable promoters is within the skill in the art. The recombinant viral vectors of the invention can also comprise inducible or regulatable promoters for expression of the siRNA in the tissue or cells where expression is desired, e.g. neuronal cells.

As used herein, an “effective amount” of the siRNA is an amount sufficient to cause RNAi mediated degradation of the target mRNA, or an amount sufficient to ameliorate Fragile X symptoms in a subject. RNAi mediated degradation of the target mRNA can be detected by measuring levels of the target mRNA or protein in the cells of a subject, using standard techniques for isolating and quantifying mRNA or protein as described above.

One skilled in the art can readily determine an effective amount of the siRNA of the invention to be administered to a given subject, by taking into account factors such as the size and weight of the subject; the extent of the disease penetration; the age, health and sex of the subject; the route of administration; and whether the administration is regional or systemic. Generally, an effective amount of the siRNA of the invention comprises an intracellular concentration of from about 1 nanomolar (nM) to about 100 nM, preferably from about 2 nM to about 50 nM, more preferably from about 2.5 nM to about 10 nM. It is contemplated that greater or lesser amounts of siRNA can be administered.

Recently it has been shown that morpholino antisense oligonucleotides in zebrafish and frogs overcome the limitations of RNase H-competent antisense oligonucleotides, which include numerous non-specific effects due to the non target-specific cleavage of other mRNA molecules caused by the low stringency requirements of RNase H. Morpholino oligomers therefore represent an important new class of antisense molecule. Oligomers of the invention may be synthesized by standard methods known in the art. As examples, phosphorothioate oligomers may be synthesized by the method of Stein et al. (1988) Nucleic Acids Res. 16, 3209 3021), methylphosphonate oligomers can be prepared by use of controlled pore glass polymer supports (Sarin et al. (1988) Proc. Natl. Acad. Sci. USA. 85, 7448-7451). Morpholino oligomers may be synthesized by the method of Summerton and Weller U.S. Pat. Nos. 5,217,866 and 5,185,444.

An example of a suitable ADAM10 siRNA is for instance the one recently described in a paper by Hurst et al. (Hurst et al., 2012). Others are commercially available.

The ADAM10 gene product inhibitor may also be an inhibitor of ADAM10 protein. A typical example thereof is an anti-ADAM10 antibody.

The term ‘antibody’ or ‘antibodies’ relates to an antibody characterized as being specifically directed against ADAM10 or any functional derivative thereof, with said antibodies being preferably monoclonal antibodies; or an antigen-binding fragment thereof, of the F(ab′)2, F(ab) or single chain Fv type, or any type of recombinant antibody derived thereof. These antibodies of the invention, including specific polyclonal antisera prepared against ADAM10 or any functional derivative thereof, have no cross-reactivity to other proteins. The monoclonal antibodies of the invention can for instance be produced by any hybridoma liable to be formed according to classical methods from splenic cells of an animal, particularly of a mouse or rat immunized against ADAM10 or any functional derivative thereof, and of cells of a myeloma cell line, and to be selected by the ability of the hybridoma to produce the monoclonal antibodies recognizing ADAM10 or any functional derivative thereof which have been initially used for the immunization of the animals. The monoclonal antibodies according to this embodiment of the invention may be humanized versions of the mouse monoclonal antibodies made by means of recombinant DNA technology, departing from the mouse and/or human genomic DNA sequences coding for H and L chains or from cDNA clones coding for H and L chains. Alternatively the monoclonal antibodies according to this embodiment of the invention may be human monoclonal antibodies. Such human monoclonal antibodies are prepared, for instance, by means of human peripheral blood lymphocytes (PBL) repopulation of severe combined immune deficiency (SCID) mice as described in PCT/EP 99/03605 or by using transgenic non-human animals capable of producing human antibodies as described in U.S. Pat. No. 5,545,806. Also fragments derived from these monoclonal antibodies such as Fab, F(ab)′2 and scFv (“single chain variable fragment”), providing they have retained the original binding properties, form part of the present invention. Such fragments are commonly generated by, for instance, enzymatic digestion of the antibodies with papain, pepsin, or other proteases. It is well known to the person skilled in the art that monoclonal antibodies, or fragments thereof, can be modified for various uses. The antibodies involved in the invention can be labeled by an appropriate label of the enzymatic, fluorescent, or radioactive type. In a particular embodiment said antibodies against ADAM10 or a functional fragment thereof are derived from camels. Camel antibodies are fully described in WO94/25591, WO94/04678 and in WO97/49805. Technologies of modifying antibodies to pass the blood-brain barrier are well known to the skilled person.

Many different ADAM10 antibodies are available in the art; inhibitory ADAM10 antibodies are envisaged for use in the methods described herein.

It has been demonstrated that the prodomain of ADAM10 is also a specific inhibitor of ADAM10 proteolytic activity (Moss M L et al., J Biol Chem. 2007; 282(49):35712-21), thus this prodomain is also envisaged herein as an ADAM10 inhibitor.

Other inhibitors of ADAM10 include, but are not limited to, peptide inhibitors of ADAM10, peptide-aptamer (Tomai et al., 2006) inhibitors of ADAM10, and protein interferors as described in WO2007/071789, incorporated herein by reference.

A well-characterized peptide inhibitor of ADAM10 that is particularly envisaged is an anti-ADAM10 peptide that contains the sequence YGRKKRRQRRRPKLPPPKPLPGTLKRRRPPQP (SEQ ID NO: 1), e.g. such as described by Marcello et al. According to yet even further particular embodiments, the anti-ADAM10 peptide is the Tat-Pro ADAM10⁷⁰⁹⁻⁷²⁹ peptide, i.e. consists of the sequence YGRKKRRQRRRPKLPPPKPLPGTLKRRRPPQP (SEQ ID NO:1). This cell-permeable peptide is obtained by linking the 11 aa human immunodeficiency virus Tat transporter sequence to the 21 aa sequence (Aarts et al., 2002) corresponding to ADAM10 proline rich domains (Marcello et al., 2007). The peptide crosses the blood-brain barrier and penetrates neurons (Marcello et al., 2007).

Small molecule inhibitors, e.g. small organic molecules, and other drug candidates can be obtained, for example, from combinatorial and natural product libraries. Several small molecule inhibitors of ADAM10 have already been described, and include for instance G1254023X (e.g. Hoettecke et al., 2010) and triptolide (e.g. Soundararajan et al., 2010). These are also commercially available. Other small molecule inhibitors include, but are not limited to, those described in Liu P et al., 2006 (Identification of ADAM10 as a major source of HER2 ectodomain sheddase activity in HER2 overexpressing breast cancer cells, Cancer Biology & Therapy, particularly FIG. 5 therein).

Other inhibitors combine inhibition of ADAM10 and ADAM17 (e.g. INCB3619, INCB7839, GW280264X). These are also envisaged herein, since they also inhibit ADAM10. However, it can be foreseen that a specific ADAM10 inhibitor will have less side (i.e. off-target) effects. Thus, particularly envisaged inhibitors are inhibitors that have a high selectivity for ADAM10 (i.e. inhibit ADAM10 with a significantly greater affinity than that they inhibit other proteins).

In summary, an “inhibitor of ADAM10” as used herein can be, but is not limited to: a chemical, a small molecule, a drug, an antibody, a peptide, a secreted protein, the ADAM10 prodomain, a nucleic acid (such as DNA, RNA, a polynucleotide, an oligonucleotide or a cDNA) or an antisense RNA molecule, a ribozyme, an RNA interference nucleotide sequence, an antisense oligomer, a zinc finger nuclease or a morpholino.

Inhibition of ADAM10 gene product does not necessarily mean complete ablation of ADAM10 function, although this is envisaged as well. Particularly with antisense RNA and siRNA, but with antibodies as well, it is known that inhibition is often partial inhibition rather than complete inhibition. However, lowering functional ADAM10 gene product levels will have a beneficial effect even when complete inhibition is not achieved—as it restores the proper balance of APP processing. Thus, according to particular embodiments, the inhibition will result in a decrease of 10%, 20%, 25%, 30%, 40%, 50%, 60%, 70%, 75%, 80%, 90% or up to 100% of functional ADAM10 gene product. According to particularly envisaged embodiments, the inhibition of Adam10 is partial inhibition, e.g. between 10 and 80%, between 20 and 70%, between 20 and 60%, between 20 and 50%, between 20 and 40%, between 30 and 50% or between 30 and 40% inhibition. Methods of measuring the levels of functional ADAM10 gene product are known to the skilled person, and he can measure these before and after the addition of the inhibitor to assess the decrease in levels of functional ADAM10 gene product.

It is particularly envisaged that administration of the inhibitor (or the use of the inhibitor in treatment) will result in (at least partial) rescue of spine dysmorphogenesis. Thus, according to particular embodiments, the treatment of Fragile X syndrome is (at least in part) rescue of the spine malformations observed in Fragile X syndrome.

It is to be understood that although particular embodiments, specific configurations as well as materials and/or molecules, have been discussed herein for cells and methods according to the present invention, various changes or modifications in form and detail may be made without departing from the scope and spirit of this invention. The following examples are provided to better illustrate particular embodiments, and they should not be considered limiting the application. The application is limited only by the claims.

### EXAMPLES

Materials and Methods

Mice and Animal Care. Animal care was conducted conforming to the institutional guidelines that are in compliance with international laws and policies (DL N116, GU, suppl 40, 18-2-1992, European Community Council Directive 86/609, OJa L 358, 1, Dec. 12, 1987; National Institutes of Health Guide for the Care and Use of Laboratory Animals, US National Research Council, 1996) and approved by the Institutional Ethical Board at the KU Leuven, Belgium. The C57BL/6 Fmr1 KO mice were described in (Bakker et al., 1994) while C57BL/6 WT were breed in house. Seven, fourteen, twenty-one, thirty and nineteen days old male mice were used in this study.

Neuronal culture preparation and stimulation. Mouse primary cortical neurons (E15) were prepared as described (Ferrari et al., 2007). Neurons were treated with the mGluRI agonist DHPG (100 uM) for 5 min as described in Napoli et al., 2008. Timp-1 (15 nM) was added to the media for 4 h.

Lymphoblastoid cell lines. Lymphoblastoid cell lines were grown in suspension in DMEM-F12 media (Invitrogen) supplemented with Fetal Bovine Serum 10% (FBS, Invitrogen) and 1% penicillin-streptomycin (Invitrogen). Cells were kept at 37° C. in 5% CO2.

Brain protein extracts. Brain were lysate in 100 mM NaCl, 10 mM MgCl2, 10 mM Tris-HCl pH 7.5, 1% Triton X-100, 1 mM dithiothreitol, 40 U ml-1 RNAse OUT (Invitrogen), 5 mM β-glycerophosphate, 0.5 mM Na3VO4, 10 μl ml-1 Protease inhibitor cocktail (PIC, Sigma). Lysates were centrifuged 8 min at 12,000 g at 4° C. The supernatant was used for immunoprecipitations and western blot. To obtain fractions enriched in membrane protein were lysate in 100 mM NaCl, 10 mM MgCl2, 10 mM Tris-HCl pH 7.5, 1% Triton X-100, 1% NaDeoxycholate, 1 mM dithiothreitol, 40 U ml-1 RNAse OUT (Invitrogen), 5 mM β-glycerophosphate, 0.5 mM Na3VO4, 10 μl ml-1 Protease inhibitor cocktail (PIC, Sigma). Extract were incubated 1 h on ice before spinning. For total protein analysis, brains or cells were homogenized in Laemmli buffer, boiled, and vortexed.

Immunoprecipitation and RT-PCR. Mouse brains were lysed and the supernatant was used for the IP according to standardized protocol (Napoli et al., 2008). FMRP antibody or purified rabbit IgGs were incubated with the Protein A Sepharose beads and with brain extract or cells for 1 hr at 4° C. After incubation with the extracts RNA was extracted (Phenol/Chloroform) and precipitated in 2.5 v ethanol. First-strand synthesis was achieved using p(dN)6 and 100 U of Superscript III (Invitrogen) and PCR was performed with GoTaq (Promega), according to the manufacturer's instructions. We used specific oligonucleotides to amplify αCaMKII, App, Adam10, Adam9, Adam17, Cyp46, BC200, D2DR. When RTqPCR was used to quantify immunoprecipitated mRNA, the relative mRNA level in the immunoprecipitation samples was calculated as follows: 2^{-[DCp(Ip)-DCp(IgG)]}=2^{-DDCp}, where DCp equals Cp(IP or IgG)-Cp(Input).

Polysomes-mRNPs analysis. For the study of mRNAs associated to polysomes-mRNPs, brains were homogenized in lysis buffer according to Napoli et al., 2008. Lysate was loaded onto a 10-60% (w/v) sucrose gradient and sedimented by centrifugation at 4° C. for 150 min at 37,000 rpm in a Beckman SW41 rotor. Each gradient was collected into 12 fractions (1-7=polysomes; 8-12=mRNPs), RNAs were extracted and precipitated. mRNAs of interest (βactin, App and Adam10 mRNAs) were quantified by RT-qPCR. Real-Time qPCR was performed using the SYBRGreen mix and a Light Cycler 480.

Synaptoneurosomes. Synaptoneurosomes were prepared by homogenization of fresh tissue in ice-cold buffer as described (Napoli et al., 2008).

Western Blot. Standard methodologies were used. 5-20 μg of total lysate or synaptoneurosomes were separated by SDS-PAGE electrophoresis and blotted on a PVDF membrane (Millipore). Membranes were incubated using specific antibodies. Rabbit aFMRP (1:1000, Ferrari et al.2007), rabbit aADAM10 (1:5000, gift from W. Annaert, K. U. Leuven), aLDH (1:000, Sigma), rabbit aAPP C-ter (1:4000, Sigma), mouse aAPP N-ter (1:1000, Millipore), rabbit sAPPbeta (1:100, IBL), rabbit aNR2A (1:1000, Upstate) rabbit asAPPalpha (1:500, IBL), mouse aVinculin (1:2000, Sigma-Aldrich), mouse aGAPDH (1:10000, Chemicon). Proteins will be revealed using an enhanced chemiluminescence kit (GE Healthcare) and the imaging system LAS-3000 (Fuji). The quantification will be done using the software ImageQuant vers. 5 (Molecular Dynamics). Further quantification was performed upon Coomassie staining of the membranes.

DNA Constucts used in this study. The pEGFP construct is commercially available (Clontech, Mountain View, Calif., USA). For RNA interference, shRNA against APP (targeting nt 538-556 of mouse APP X59379.1) cloned into the pLentiLox3.7 (pLL3.7) vector has been described (Hoe et al., 2009). This shRNA target sequence is specific for mouse/rat APP since it is not found in other APP family members or in human APP. The two constructs were kindly provided by Dr. Daniel Pak (Neuroscience 2010 Aug. 11; 169(1):344-56).

Transfection. Neurons were transfected with the different constructs at 8 days in culture using the calcium phosphate method. 5 μg of DNA were mixed with 250 mM CaCl2 and added to the same volume of 2× HEPES-buffered saline (HBS). The DNA mix is incubated for 20 min and then added to the neurons for 30 min. Neurons were washed with NB and cultured in the original medium at 37° C., 5% CO2. After 6 days, neurons were fixed with paraformaldehyde 4%.

Immunofluorescence. DIV 14 neurons were fixed with paraformaldehyde (4%), permeabilized with Triton X-100 (0.2%), and analyzed for APP (C-ter Sigma) and ADAM10. Cells were then incubated with and Alexa488 Goat anti-rabbit secondary antibody (1:1000, Invitrogen). To detect surface APP, cells were labelled prior fixation at 4 C using Ab against APP (N-ter). After washing, neurons were mounted on slides using Fluoromount (Sigma). Controls staining were performed omitting primary antibodies.

Image Acquisition and Quantification. Confocal images were obtained using a confocal laser scanning microscope (Nikon) 40× oil objective. Each image has 2048×2048 pixels resolution and is a z series projection of approximately 7 to 15 images taken at 0.5 um depth intervals. Labelled transfected pyramidal neurons were chosen randomly for quantification. Quantitative analysis was performed blind from three different cultures. For each neuron a total of 4 dendrites over a length of 20 μm and starting 50 μm from cell body were analyzed. Morphometric measurements were performed manually using Image J software (1.44p version).

Biotinylation of Cell Surface Proteins. Neurons were incubated with Sulfo-NHS-LC-Biotin (0.2 mg/ml in PBS pH 7.9) for 30 min at 4° C. Excess biotin was quenched with 0.2 M glycine for 30 min. Cells were lysate in STEN buffer and immunoprecipitated with streptavidin beads (Invitrogen) 30′ 4° C. Samples were analyzed by Western Blot.

ABeta 40-42 ELISA. Brain were be solubilized in Gu-HCl and Abeta was detected using sandwich ELISA assay according to the manufacturer's specifications (The Genetics Company).

Brain fractionation. Half brain was homogenized in STE buffer (Sucrose 0.32 M, 5 mMTris HCl pH 7.4, 1 mM EDTA, PSMF 0.1 mM, 10 μl ml⁻¹ Protease inhibitor cocktail (PIC, Sigma). The homogenized tissue was centrifuged at 800 g for 10 min to pull down nuclei. The resulting supernatant was then centrifuged at 55000 rpm 1 h. The supernatant (S1) was collected while the pellet was dissolved in 1% Triton STE Buffer and centrifuged at 55.000 rpm (TLX) for 1 hr. The supernatant is collected (S2) and the final pellet homogenized in SDS Laemmli buffer 1× (Triton-insoluble fraction, P1).

Peptide treatment. Specific (TAT-PRO) or control (TAT-ALA) peptides (10 μM) were added to the media of DIV14 WT and KO cultured neuron and the release of sAPP was monitored by WB after 24 h. P20 mice were given intraperitoneal injections of the two peptides (3 nmol/g) and fractionation performed after 24 hrs. Experiment was conducted on WT and KO littermate mice.

Morphological analysis. Wild type and Fmr1 KO mice received an intraperitoneal injection with either TAT-Pro or TAT-Ala peptides (3 nmol/g; 14-d-old mice; 8 mice/group). After 24 h, the mice were deeply anaesthetised with an intraperitoneal injection 0.54 mg/g Avertin and then perfused with ice-cold 4% PFA in 0.1M phosphate buffer (PB; 61.2 mM Na2HPO4, 23.2 mM NaH2PO4.H20, pH 7.4). Brains were removed and sectioned in 200 μm thick transverse slices containing both cortex and hippocampus using a Vibratome (Vibratome). Slices were equilibrated in PBS at 4° C. before applying the Dil-coated particles using a diolistic gene-gun system (Bio-Rad laboratories, Italy). 15 min after application, slices were fixed with 4% PFA in 0.1M PB (2 hours) and kept in PB O/N before mounting. Images of Dil-labelled neurons in the hippocampus and cortex were acquired using Zeiss Confocal LSM510 Meta system with 63× objective and a sequential acquisition setting at 1024×1024 pixels resolution. For each image three to four 0.5 μm sections were acquired and z-projection was obtained. Morphological analysis of dendritic spines was performed using freely available ImageJ 1.42q software (Wayne Rasband National Institutes of Health, USA), and spine length, head width and neck width were measured.

Electrophysiology. Animals were euthanized by decapitation, and the right hippocampus was rapidly dissected out into ice-cold (4° C.) artificial cerebrospinal fluid (ACSF) and saturated with carbogen (95% O₂/5% CO₂). The ACSF consisted of (in mM): 124 NaCl, 4.9 KCl, 24.6 NaHCO₃, 1.20 KH₂PO₄, 2.0 CaCl₂, 2.0 MgCl₂, and 10.0 glucose, pH 7.4. Transverse slices (400 μm thick) were prepared from the dorsal area of the hippocampus with a tissue chopper and were placed into a submerged-type chamber; samples were kept at 33° C. and continuously perfused with carbogen saturated ACSF at a flow-rate of 2.2 ml/min. After 90 min of incubation, one slice was arbitrarily selected, and a tungsten electrode was placed in the CA1 stratum radiatum. Field excitatory postsynaptic potentials (fEPSPs) were recorded by placing a glass electrode (filled with ACSF, 3-7 MΩ) in the stratum radiatum opposite the stimulating electrode. The time course of the field EPSP was measured as the descending slope function for all sets of experiments. After input/output curves had been established, the stimulation strength was adjusted to elicit a fEPSP-slope of 35% of the maximum and kept constant throughout the experiment. During the baseline recording, 3 single stimuli (0.1 ms pulse width; 10 s interval) were measured every 5 min. The mGluR mediated LTD was induced by a bath application of 30 μM S-DHPG [(S)-3-5-dihydroxy-phenyl glycine; Abcam Chemicals Ltd., Bristol UK] for 15 min (Tambuyzer et al., 2013). During the chemical induction, evoked responses were monitored at 1, 4, 7 and 10 min and then every 5 min until the end of the experiment. All studies were conducted with the experimenter blinded to the treatment regime.

Behavioral tests. All behavioral experiments were performed with P24-25 male mice and blind to the genotype and treatment. We used a minimum of 9 animals per genotype and treatment. Mice were habituated to their new environment for 3 days and tests were conducted during the light phase of their activity cycle. Health and weight of the mice were recorded routinely during the whole period of testing.

Open-field exploration was tested in 50 cm×50 cm×30 cm (w×l×h) square arena illuminated by indirect light. Animals were dark adapted for 30 min and placed in the arena for 10 min each. Movements of the mice in the arena were video-tracked for 10 min and the images were transmitted to a PC equipped with ANY-maze™ Video Tracking System software (Stoelting Co., IL, USA).

Spatial working memory was evaluated in a delayed nonmatching alternation test using an elevated open T-shaped maze made of dark grey plastic. The maze was composed of three 50 cm×7 cm×4 cm (l×w×h) arms elevated 25 cm above the ground. Behavior was registered with a PC-interfaced camera and analyzed with ANY-maze™ Video Tracking System software (Stoelting Co., IL, USA). The mouse was subjected to two trials. During the exploration trial, one of the horizontal T-maze arms (novel arm) was blocked with a 10 cm high door and the mouse was placed into the start arm to explore freely the start and familiar arms for 5 min. During the test trial 1 h later, the mouse was placed again into the start arm to explore the maze with all arms accessible. The maze was cleaned between trials to avoid confounding odor or food traces.

### Example 1

### Example 1

### APP is Highly Expressed in Fragile X Syndrome

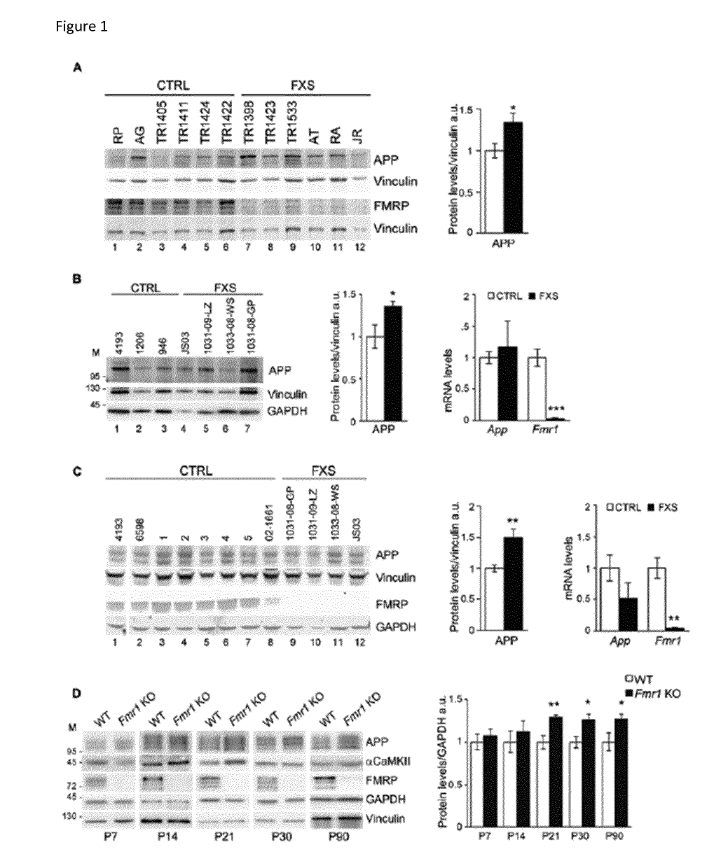

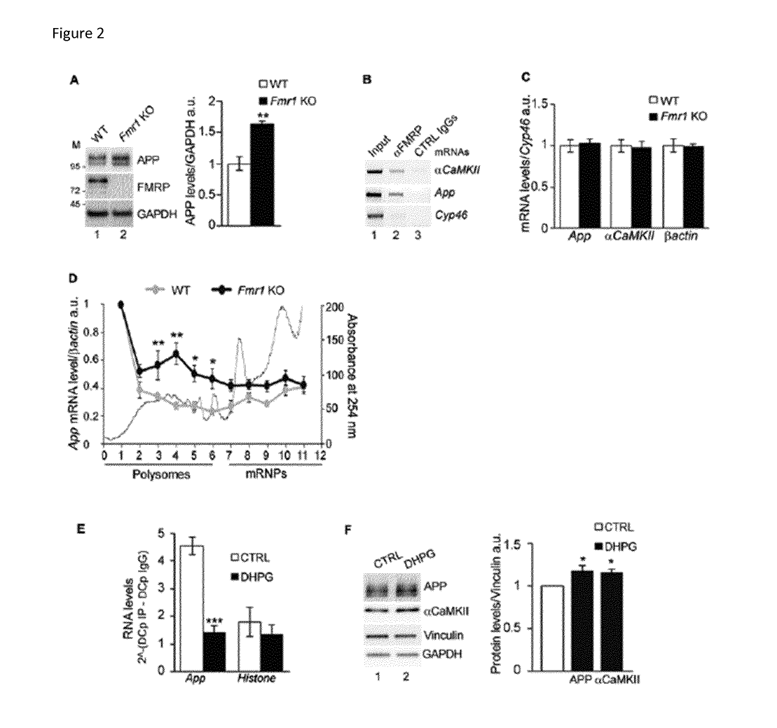

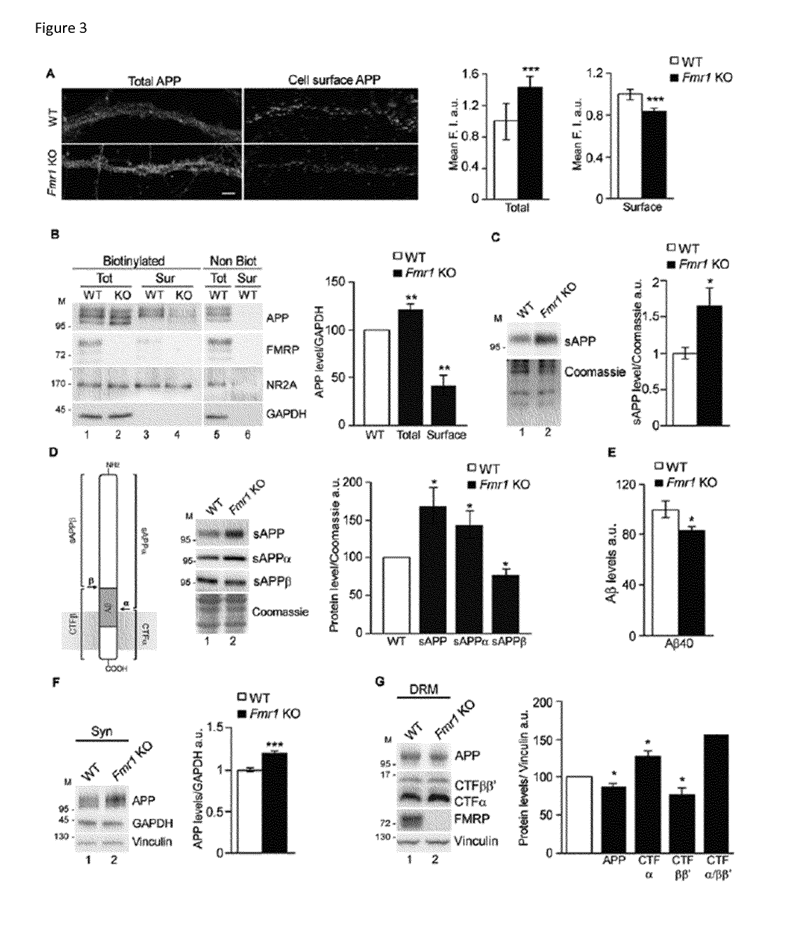

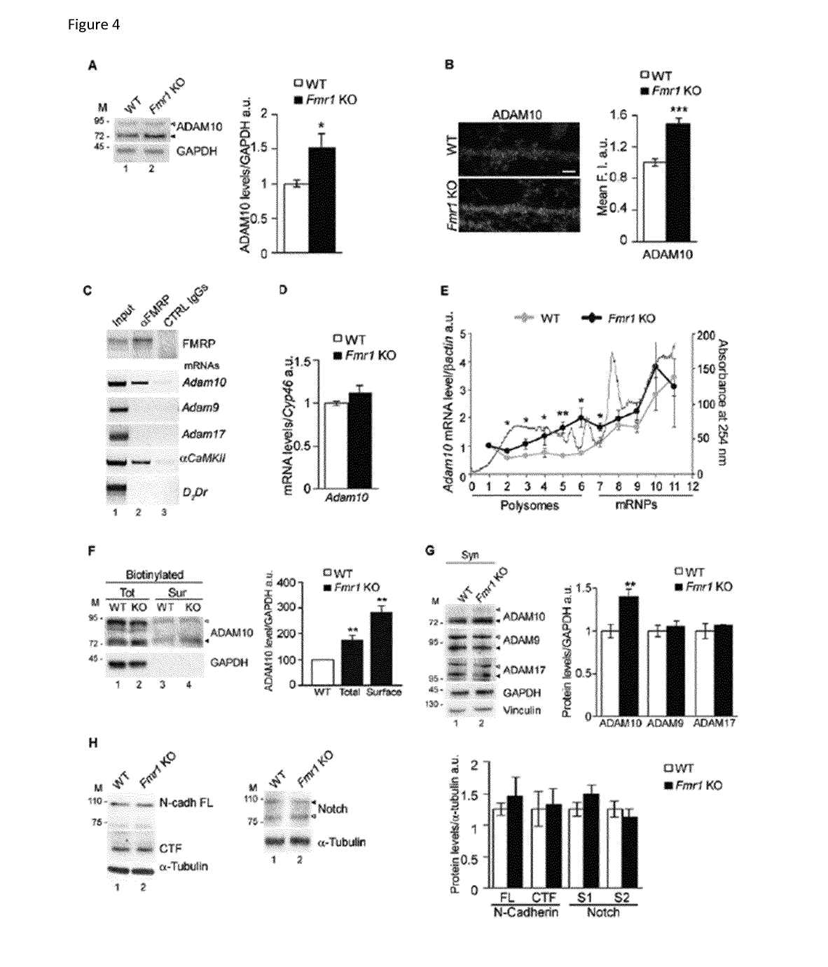

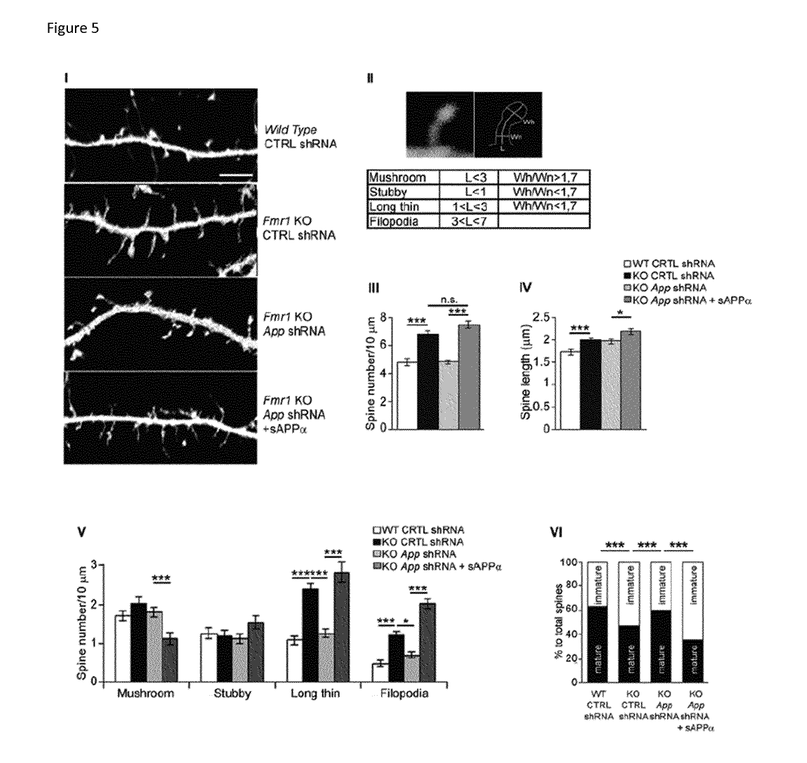

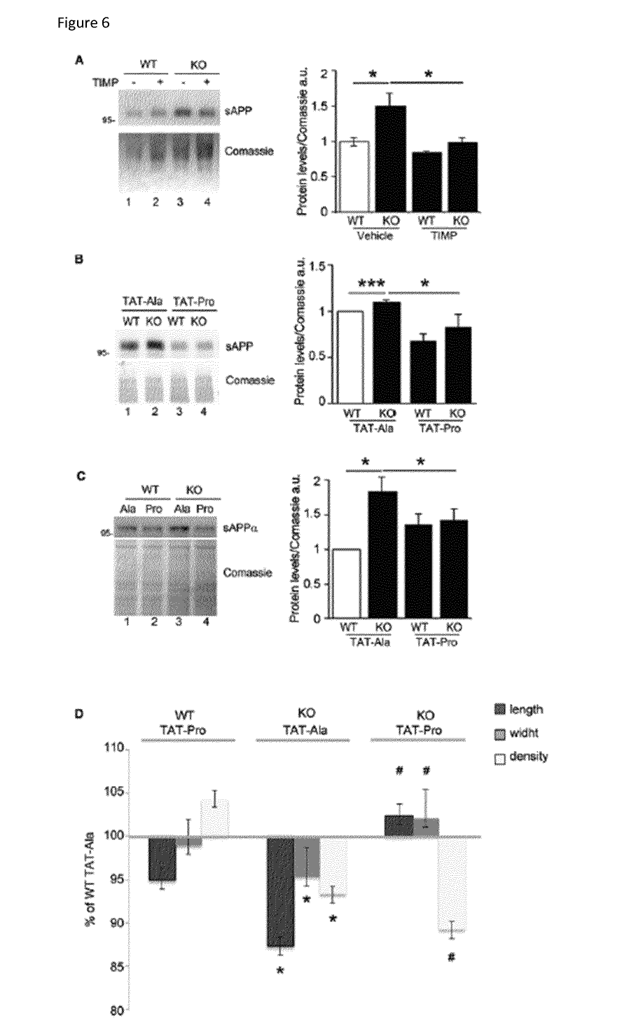

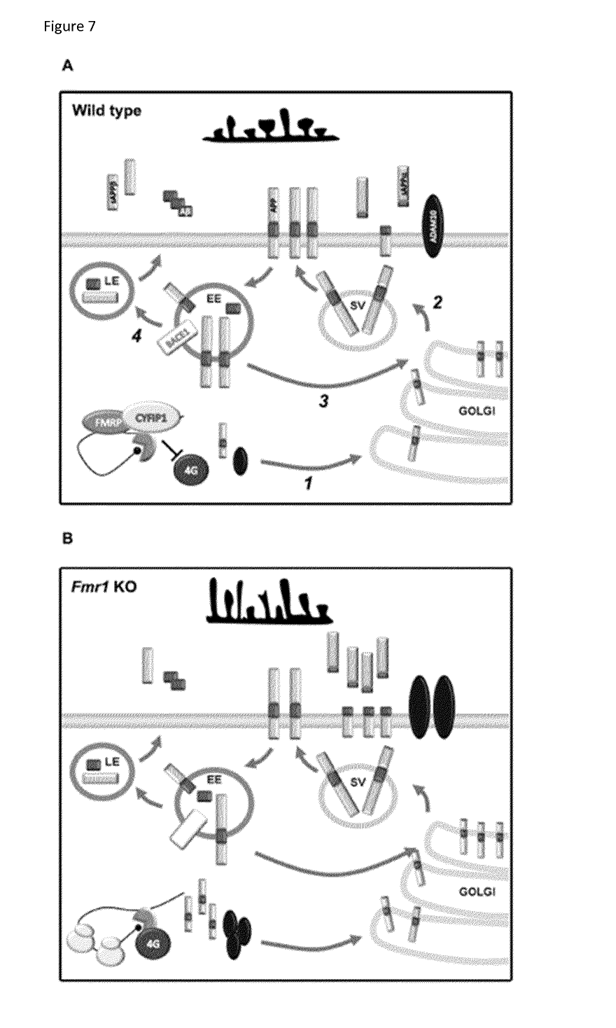

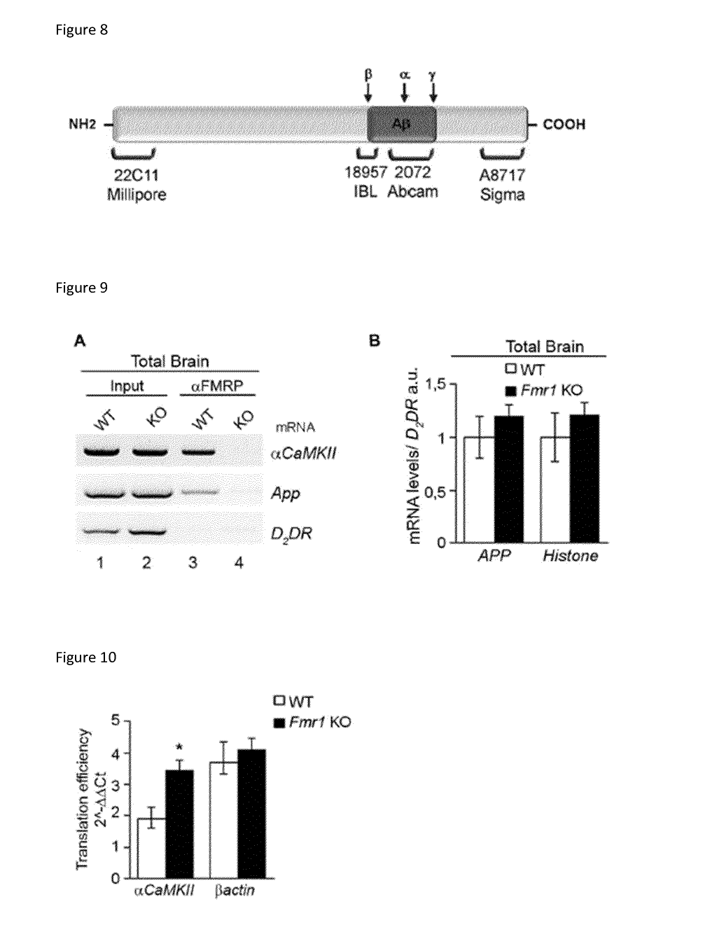

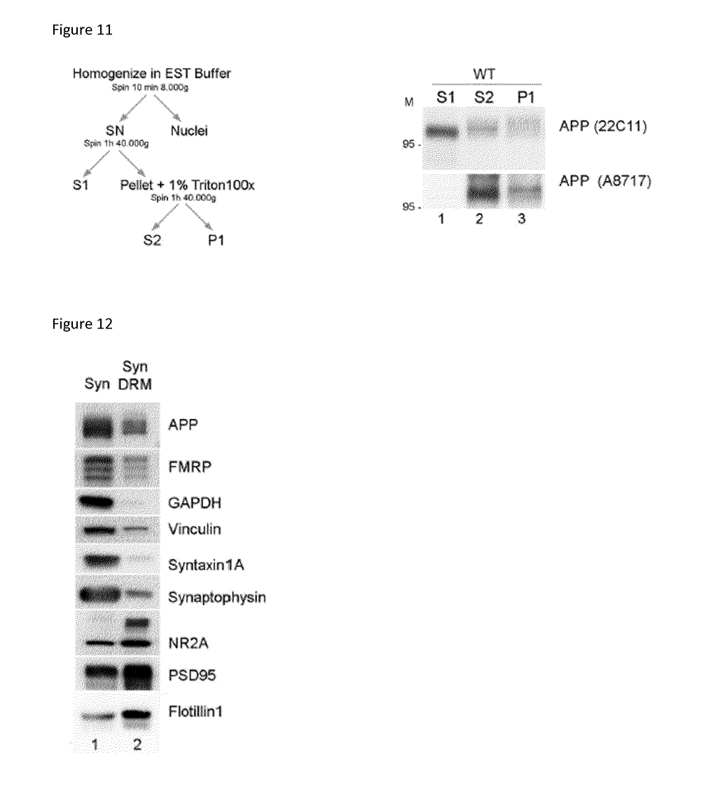

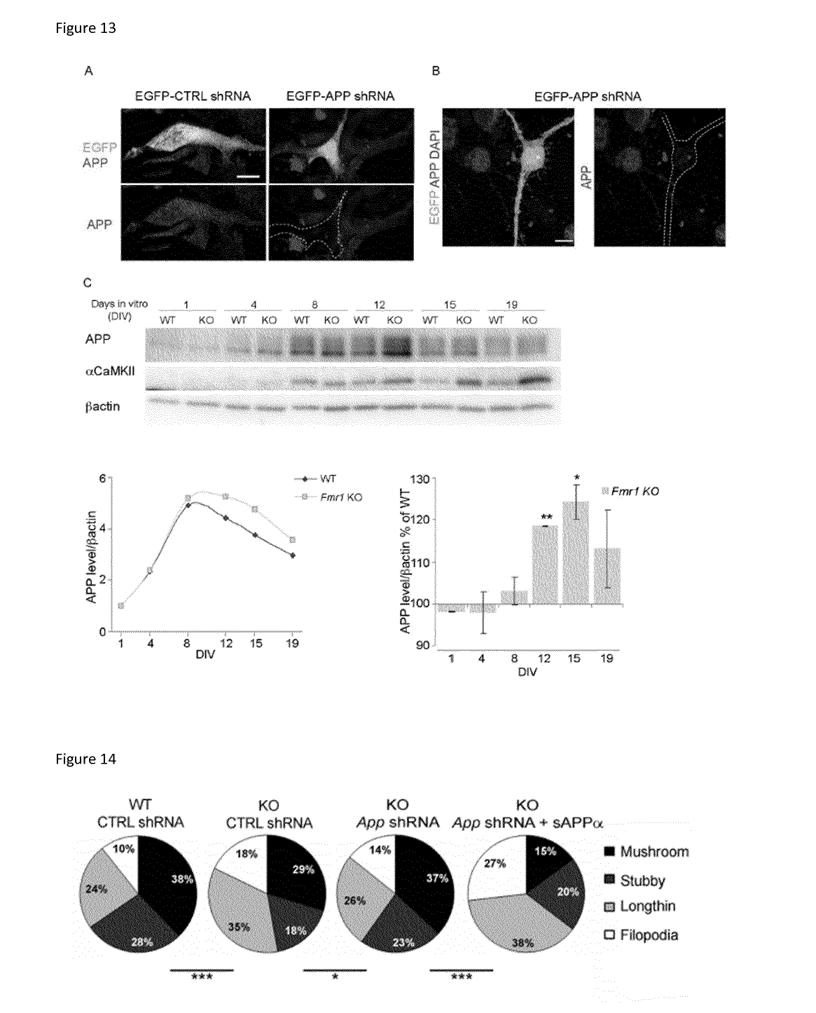

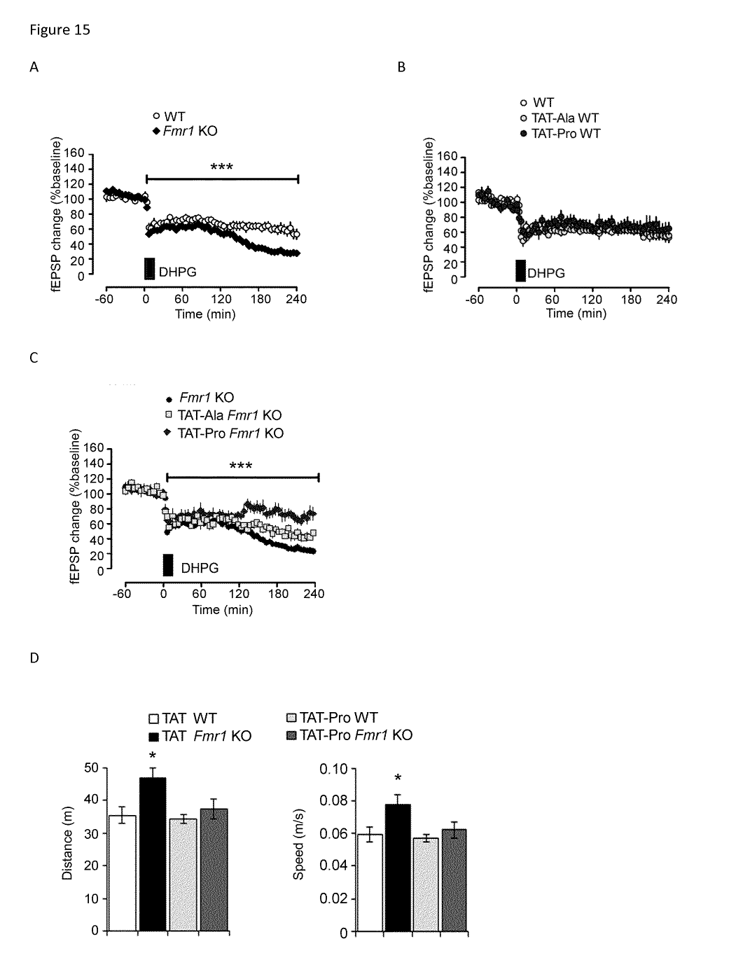

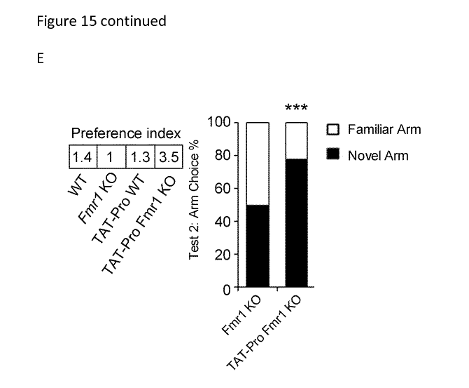

App mRNA was recently described as target of the FMRP-CYFIP1 translational inhibitory complex (Napoli et al., 2008). Consistently, APP levels are increased when the levels of FMRP (Westmark and Malter, 2007) or CYFIP1 are decreased (Napoli et al., 2008). APP is highly expressed in Lymphoblastoid cell lines from Fragile X patients when compared with controls (FIG. 1A). In addition, we analysed human post-mortem samples and found that absence of FMRP affects APP expression (increase) in both Cortex and Cerebellum (FIG. 1B-C).

Since FXS is a neurodevelopmental disorder, we first wanted to investigate whether APP deregulation in the absence of FMRP occurs at specific stages of postnatal development. Whole brain extracts from Fmr1 KO and wild type (WT) mice, during postnatal development (P7, 14, 21) and adulthood (P30 and P90), were analyzed using an antibody against the C terminus of APP (A8717, FIG. 8). No differences in APP expression were observed between WT and KO mice at P7 and P14. APP was significantly upregulated in Fmr1 KO mice three weeks after birth and during adulthood (FIG. 1D). αCaMKII levels were also upregulated in young Fmr1 KO animals, as previously reported (Hou et al., 2006; Lu et al., 2004; Zalfa et al., 2003), but downregulated at P7, suggesting that αCaMKII mRNA is differentially regulated by FMRP in a stage-dependent manner. GAPDH and Vinculin did not change in the absence of FMRP. These findings suggest that APP is developmentally regulated by FMRP.

Immunoprecipitation experiments of the FMRP complex in brain showed that App mRNA is associated to FMRP (FIG. 9A). The steady-state levels of App mRNA did not change between WT and Fmr1 KO animals (FIG. 9B), in agreement with previous data (Zalfa et al., 2007), showing that FMRP does not alter App mRNA stability.

These results suggest that FMRP might regulate App mRNA rate of translation. This effect starts at three weeks after birth, which corresponds to a phase of synaptic consolidation.

### Example 2

### FMRP Modulates App mRNA Translation

To further extend the analysis of FMRP-mediated regulation of APP, we focused on brain cortices of three weeks old animals (P21). In this area, APP is highly expressed and was described to affect spine morphology (Bittner et al., 2009; Lee et al., 2010b). Consistently with our findings from human and mouse brain (FIG. 1D), APP was upregulated in cortices from P21 Fmr1 KO mice (FIG. 2A) where the effect of such a dysregulation appears to be stronger (1.64+/−0.04 vs 1.29+/−0.07). As in total brain, App as well as αCaMKII mRNA associated with FMRP (FIG. 2B), as shown by FMRP immunoprecipition from cortical extracts followed by RT-PCR. Additionally, consistent with the findings of total brain, in cortex the level of App mRNA was not affected by the absence of FMRP (FIG. 2C).

To directly assess if FMRP controls APP protein synthesis, translationally active polysomes and silent mRNPs were fractionated from mouse cortex and the mRNAs distribution was analyzed. As shown in FIG. 2D, App mRNA was co-fractionating more with polysomes in Fmr1 KO cortices compared to WT. αCaMKII mRNA, known to be translationally regulated by FMRP (Hou et al., 2006; Zalfa et al., 2003), was also more abundant in the polysomal fraction (FIG. 10). βactin mRNA does not change its translation in FXS (Lee et al., 2010a; Zalfa et al., 2003). This evidence indicates that FMRP represses App mRNA translation.

It has been widely shown that FMRP-dependent translation responds to stimulation of group I metabotropic glutamate receptors (mGluR) (Bassell and Warren, 2008; Dolen et al., 2007; Ferrari et al., 2007; Huber et al., 2002; Weiler et al., 1997). Upon mGluRs activation, FMRP releases some target mRNAs triggering their translation (Gross et al., 2012; Liu-Yesucevitz et al., 2011; Napoli et al., 2008). In agreement with these findings, we found that application of the group I mGluR agonist DHPG to primary cortical neurons reduced by threefold the amount of App mRNA associated with FMRP (FIG. 2E) and induced an increase in the expression of APP (FIG. 2F). Similar findings were observed for αCamKII, a well-known regulated FMRP target (Zalfa et al., 2003; Hou et al., 2006).

All together, these data demonstrate that App mRNA is associated with FMRP, which represses its translation; moreover, the mGluRs pathway tunes the FMRP-App mRNA complex.

### Example 3

### The Absence of FMRP Reduces the Levels of Cell-Surface APP in Neurons

To investigate if increased APP production also leads to an increased insertion in the plasma membrane, we tested surface levels of APP on cortical neurons (DIV 14) from WT and Fmr1 KO mice. We stained total or surface APP in neurons using an antibody against APP intracellular domain or ectodomain (FIG. 8). We compared the fluorescent intensity in WT and Fmr1 KO. Surprisingly, while total APP levels were increased in KO primary neurons, consistent with our findings in brain extracts, surface APP was dramatically decreased in FMRP-lacking cells (FIG. 3A). To further monitor the cell surface APP, proteins were biotinylated with sulfo-NHS-LC-Biotin, captured using streptavidin-dynabeads and analyzed by Western blot (FIG. 3B). NMDA receptor subunit NR2A, whose levels are unaffected in Fmr1 KO (Giuffrida et al., 2005; Li et al., 2002) were unchanged between WT and Fmr1 Ko cell-surface proteins. Even though total APP levels were increased in KO (compare lanes 1 and 2), surface APP was decreased (compare lanes 3 and 4). Streptavidin pull downs in the absence of cell-surface protein biotinylation revealed no signal (FIG. 2B, compare lanes 5 and 6).

These findings suggest that the absence of FMRP not only affects APP protein synthesis, but also its proteolysis and/or its delivery to the cell surface.

### Example 4

### APP Processing is Impaired in Fmr1 KO Brain

To address whether the decreased APP on the surface was due to increased cleavage, we investigated the amount of soluble APP (sAPP) released by the cells. As shown in FIG. 3C, sAPP was indeed increased in Fmr1 KO neurons. To directly test whether this was specifically due to defective activity of α-secretase, we measured the levels of secreted APP (total sAPP, sAPP α, sAPP β) in brain from young WT and KO mice (FIG. 3D) through a fractionation method (FIG. 11). Using an antibody against APP C-terminus (FIG. 8). The soluble fraction was enriched in sAPP, while the full length protein was absent (FIG. 11). Using specific antibodies to discriminate total sAPP, sAPP α and sAPP β we found that the rise in sAPP levels is caused by increased α-cleavage; β-cleavage was slightly decreased (FIG. 3D, central and right panel). Finally, Aβ40 generation was slightly but significantly reduced in absence of FMRP (FIG. 3E).

All together, these findings show that in young Fmr1 KO animals the a-cleavage is enhanced, while β-cleavage is reduced.

Since spine morphology and functionality are compromised in FXS, we investigated whether FMRP loss alters APP production and α-cleavage also at synapses. First, we found that APP is highly expressed at synapses (FIG. 3F). Notably, APP was found slightly reduced in Fmr1 KO mice in detergent resistant microdomains (DRMs) fractionated from synaptoneurosomes (FIG. 3G, FIG. 12). To prove that these effects were due to enhanced α-cleavage, we measured the abundance of individual CTF isoforms in those preparations. As predicted, in the absence of FMRP, CTFα was increased, while CTFβ and CTFβ′ were decreased (FIG. 3G). CTFβ and CTFβ′ are both products of β-secretase (Zhou et al., 2011). The increased CTF α/CTF-ββ′hu 3 1³′ ratio (FIG. 3G) confirms that α-secretase activity is specifically upregulated in young Fmr1 KO animals.

### Example 5

### ADAM10 is Upregulated in the Absence of FMRP

To identify the mechanism behind the increased sAPPα release in the absence of FMRP, we decided to measure the expression levels of ADAM10, the constitutive APP α-secretase. Western blotting (FIG. 4A) and immunofluorescence for ADAM10 in cortical lysates and primary neurons (FIG. 4B) showed an increased ADAM10 expression in absence of FMRP.

Adam10 mRNA bears a G-quartet sequence embedded in a G-rich region in its 5′UTR, similar to the one found in the coding region of App mRNA (Westmark and Malter, 2007). This region is conserved in primates and rodents and was recently implicated in Adam10 mRNA translational inhibition (Lammich et al., 2010; Lammich et al., 2011). Since FMRP binds target mRNAs also through G-rich and G-quartets and regulates mRNA translation (Bagni and Greenough, 2005; Bassell and Warren, 2008), we reasoned that ADAM10 upregulation at synapses might be due to a direct effect of FMRP on Adam10 mRNA metabolism. To test this hypothesis, FMRP was immunoprecipitated from brain extracts and the bound mRNA analyzed by RT-PCR. The specificity of the immunoprecipitation was verified by the absence of bound mRNA using non-specific IgGs, as well as by the absence of D₂DR mRNA (FIG. 4C), known to be absent in the FMRP complex (Centonze et al 2007 J Neurosci). Notably, Adam10 mRNA was found to be associated with FMRP, as well as the known FMRP mRNA target, αCaMKII mRNA. ADAM9 and ADAM17 were shown to contribute to a-cleavage in vitro, (Vingtdeux and Marambaud, 2012), no association with Adam9 or Adam17 mRNA was detected, showing the specificity of FMRP-Adam10 mRNA interaction (FIG. 4C).

Since Adam10 mRNA steady-state levels did not significantly change between WT and KO mice (FIG. 4D), we investigated if Adam10 mRNA was translationally dysregulated. As shown in FIG. 4E, Adam10 mRNA is more associated with actively translating polysomes in KO compared to WT animals, thus causing increased ADAM10 expression in absence of FMRP. Moreover, at the cell-surface more mature ADAM10 was detected (FIG. 4F). Interestingly, ADAM10 mainly generates sAPPα at the cell surface (Hartmann et al., 2002; Jorissen et al., 2010; Kuhn et al., 2010; Lammich et al., 1999; Lichtenthaler, 2011). We then studied the synaptic expression of the different metalloproteases. As shown in FIG. 4G there is a selective increase of ADAM10 but not ADAM9 or 17 in Fmr1 KO compared to WT mice.

All together these data demonstrate that FMRP represses Adam10 and App mRNA translation in cortex.

### Example 6

### ADAM10 Dysregulation in Young Fmr1 KO Mice Selectively Affects APP Processing

To determine whether increased ADAM10 expression in three weeks old Fmr1 KO mice could result in a general dysregulation the generation of cleavage protein products mediated by ADAM10, were quantified by Western blotting from WT and KO cortices. Three other well-described ADAM10 targets such as Notch, N-cadherin, Ephrin were analyzed (FIG. 4H and data not shown). Of note, only APP shedding was increased, highlighting an important role for FMRP in coordinating APP expression and processing in three weeks old animals. However we cannot exclude that a dysregulated processing of additional proteins could occur at other developmental stages.

sAPP α Dysregulation Contributes to the Spine Dysmorphogenesis Observed in Fmr1 KO Neurons

Evidence suggests that both APP and sAPP a affect synaptogenesis (Lee et al., 2010b; Wang et al., 2009; Zhang et al., 2011; Zheng and Koo, 2011), as well as maintenance (Lee et al., 2010b) and differentiation of synapses (Torroja et al., 1999). We wondered whether the increased APP and sAPPα might contribute to the spine dysgenesis observed in FXS. Therefore, we manipulated APP expression in Fmr1 KO neurons and analyzed the effect on dendritic spine density and morphology.

First, we downregulated APP in Fmr1 KO neurons using a lentiviral vector (Lee et al., 2010b) carrying a short hairpin RNA directed against App mRNA (App shRNA-EGFP), or a scrambled sequence (CTRL shRNA-EGFP). WT and Fmr1 KO mouse cortical neurons were transfected at DIV8, when APP starts to be highly expressed (FIG. 13C). The specificity of APP silencing was assessed using transfected MEFs and primary neurons (FIG. 13A, 13B). Furthermore, to address the role of sAPP α neurons knocked down for APP were treated with sAPPα after transfection with shRNA, according to Copanaki et al., 2010. In all conditions, dendritic spine density and morphology of pyramidal neurons were analyzed at DIV14, as described in FIG. 5.

These analysis showed that Fmr1 KO neurons have more abundant spines compared to WT (FIG. 5-I and 5-III), confirming previous observations (Bagni and Greenough, 2005; Cruz-Martin et al., 2010; Pfeiffer and Huber, 2009). Moreover, we found that APP knockdown in Fmr1 KO neurons restored normal spine density and importantly, sAPPα treatment abolished this rescue (FIG. 4-III). All together, these data indicate that the defects in spine density observed in KO neurons are caused by the excess of APP and sAPPα. Moreover, Fmr1 KO neurons display longer spines and of note, APP knockdown failed to rescue normal spine length, possibly due to sAPPα secreted by the non-silenced neurons. Indeed, addition of sAPPα further increased spine length (FIG. 5-IV).

To investigate if excessive APP and sAPPα contribute to the spine defects in KO by selectively affecting some spine types and not others, we studied the neuronal spine morphology in WT, KO, KO App shRNA and KO App shRNA+sAPPα neurons. Mean spine head and length measures were used to cluster the spines in four classes: mushroom, stubby, long thin, and filopodia (FIG. 5-II). Mushroom and stubby are considered mature spines, while long thin and filopodia immature types.

Notably, spine distribution along the four morphological classes significantly differs between WT and KO neurons, the latter showing more long thin spines and filopodia (FIG. 5-V, FIG. 14). Noteworthy, no changes in the density of stubby and mushroom-like spines were observed between WT and KO (FIG. 5-V).

APP knockdown in KO neurons rescued the overall spine distribution (FIG. 14). The density of long thin and filopodia resembled the WT condition (FIG. 5-V), while no differences were observed in the populations of stubby and mushroom. This confirms that APP knockdown selectively affected spine types altered in KO neurons (immature spines).

Moreover, sAPPα administration in APP-depleted KO neurons significantly affected the density of three spine classes, with more long thin and filopodia and less mushroom spines (FIG. 5-V).

Further, the analysis of the mature/immature spines ratio confirmed that KO neurons have a reduced ratio compared with WT and revealed that APP reduction in KO neurons dampens this defect.

Application of sAPPα in APP-depleted KO neurons counteracts the beneficial effects of APP reduction and worsens again the mature/immature ratio (FIG. 5-VI).

In conclusion, these data support the hypothesis that concomitant deregulation of both APP expression and processing during early stages of development contributes to the spine dysmorphogenesis in FMRP-lacking neurons. A model for the role of FMRP in modulating APP metabolism at synapses is provided in FIG. 7.

### Example 7

### Inhibition of ADAM10 Activity Reduces sAPPalpha Release and Rescues the Pathological Spines Phenotype in Fmr1 KO Mice

First we checked if TIMP-1, an inhibitor of metalloproteinases (Amour et al., 2000; Brew et al., 2000), would affect sAPPα release in cultured neurons. As shown in FIG. 6A, TIMP-1 treatment (15 nm for 4 h) significantly reduces sAPP release in the medium of Fmr1 KO cortical neurons to the same extent as that of TAT-Pro peptide.

Because of the excessive APP and ADAM 10 in FXS, we explored whether the inhibition of ADAM10 activity could ameliorate spines dysmorphogenesis in vivo.

To inhibit ADAM10 activity in vivo, we used the cell permeable peptide Tat-Pro that consists of the 11 aa Tat transporter sequence linked to the ADAM10 proline-rich domains (Marcello et al., 2007). TAT-Pro crosses the blood-brain barrier, penetrates into cells and interferes with ADAM10/SAP97 interaction. The impairment of SAP97-mediated ADAM10 forward trafficking affects ADAM10 synaptic localization and, thereby reduces ADAM10 shedding activity towards APP (Marcello et al., 2007; Epis et al., 2010; Marcello et al., 2013).

The efficacy of the cell permeable peptide was first assessed in WT and Fmr1 KO primary cortical neurons, which were treated with either TAT-Pro or the control inactive Tat-Ala peptide (in which all proline residues were substituted with alanine). TAT-Pro treatment (10 μM, 1h) significantly decreases sAPP release in the medium of Fmr1 KO cortical neurons, when compared to TAT-Ala exposed neurons (FIG. 6B).

The TAT-Pro peptide was next tested in vivo. Young mice (P14) received a single intraperitoneal injection of either TAT-Pro (3nmol/g) or TAT-Ala (3 nmol/g) peptides and were sacrificed after 24h. Western Blot analyses showed a significant reduction of sAPPα levels in TAT-Pro treated Fmr1 KO mice when compared with TAT-Ala treated Fmr1 KO mice (FIG. 6C). These results demonstrate the TAT-Pro capability of rebalancing the increased sAPPα release of Fmr1 KO mice. Morphological analysis of dendritic spines was performed using a diolistic gene-gun system to propel Dil-coated particles into hippocampal sections of fixed brain tissue of TAT-Pro and TAT-Ala treated mice. As shown in FIG. 6D, TAT-Pro treatment significantly increases spines length and width and decreases spines density in TAT-Pro treated Fmr1 KO mice when compared to TAT-Ala Fmr1 KO mice. These outcomes suggest that TAT-Pro treatment efficiently modifies spines morphology rescuing the pathological phenotype in Fmr1 KO mice. Next, it was evaluated whether this altered morphology also resulted in beneficial behavioural effects.

### Example 8

### Tat-Pro ADAM10⁷⁰⁹⁻⁷²⁹ Peptide Normalizes Enhanced Hippocampal mGluR-LTD, Memory and Hyperactivity in Fmr1 KO Mice

Because one of the hallmarks of FXS is the enhanced mGluR-dependent LTD observed in the Fmr1 KO mice (Huber et al., 2002), and ADAM10 activity affects LTD (Marcello et al., 2013; Musardo et al., 2013; Prox et al., 2013), we examined whether the reduction of ADAM10 activity could ameliorate the mGluR-LTD responses in Fmr1 KO mice. The effects of the Tat-Pro ADAM10⁷⁰⁹⁻⁷²⁹ peptide on DHPG induced LTD in WT and Fmr1 KO mice were tested in juvenile (P18-25) mice. Field excitatory postsynaptic potentials (fEPSPs) were recorded from CA1 fibers in hippocampal slices in response to Schaffer collateral fibers stimulation 24 h after treatment. A bath application of DHPG (30 μM, 15 min) induced a robust LTD of fEPSPs in the WT slices (240 min: 53±9% of baseline, n=7). In agreement with previous reports (Nosyreva and Huber, 2005) we observed an enhanced DHPG-induced LTD in the Fmr1 KO mice (240 min: 28±3%, n=12) (FIG. 15A). Exposure to Tat-Pro ADAM10⁷⁰⁹⁻⁷²⁹ or Tat-Ala peptides had no detectable effect on the ability of DHPG to elicit LTD in the WT slices (65±8%, n=7 and 56±5, n=6%, respectively) when compared with the untreated control (FIG. 15B). In contrast, we found that treatment with the Tat-Pro ADAM10⁷⁰⁹⁻⁷²⁹ peptide was sufficient to prevent the enhanced LTD in the Fmr1 KO mice, indicating that the exaggerated late LTD (>2 h) in these mice (FIG. 15C) is a direct consequence of increased ADAM10 activity.

Finally, we monitored the effect of the peptide on short-term (working) memory (T-maze) and hyperactivity (open field), two behavioural features that have been consistently found altered in FXS mice (Santos et al., 2014). Fmr1 KO mice move faster and walk longer distance in the open field, and they fail more in the spontaneous alternation in the T-maze (Santos et al., 2014). Targeting ADAM10 trafficking with the Tat-Pro peptide partially ameliorates the behavioural deficits observed in the Fmr1 KO mice (FIG. 15D, E).

Together, these results demonstrate that blocking SAP97-mediated ADAM10 trafficking to the synapses reduces sAPPα production and reverses molecular, cellular and behavioural impairments that constitute the hallmark of FXS.

Discussion

Pathological Behaviour of APP in FXS

Amyloid precursor protein (APP), the parental molecule of neurotoxic amyloid-beta peptide (Ab), is a transmembrane protein extensively associated to Alzheimer's disease (AD) and recently implicated in synapse formation and synaptic plasticity (for a recent review Bordji K et al., 2011). Cleavage of APP by β- and γ-secretases release neurotoxic peptides, including Aβ, whose accumulation is directly linked to the pathogenesis. When APP is processed alternatively via the nonamyloidogenic (a-secretase) pathway, the secreted alpha form of APP (sAPPa) is produced. sAPPα has growth factor properties and promotes neurogenesis, cell proliferation and migration: all cellular events that could contribute to the underlying macrocephaly in FXS (Chiu et al., 2007). APP undergoes a tightly regulated trafficking and processing and, through either the full-length protein and/or its cleavage products, it mediates synaptogenic, neuroprotection (Kögel D, et al 2012) and synapotrophic (Mucke L, et al 1994; Seeger G, et al 2009) activities in development and during aging (Kögel D, et al 2012)). As such, it is reasonable to speculate that misregulation of APP could contribute to the neuronal and synaptic impairment occurring in AD (Zhang and Koo, 2011) and possibly in other pathologies such as Fragile X and Autism where the levels of APP were found increased (for a recent review see Sokol et al., 2011).

Increasing evidence shows that APP has also a non-pathological function at synapses. APP is present in distributed in presynaptic terminals and growth cones (Kins et al., 2006) as well as in dendrites and at post-synapses (Hoe et al., 2009). APP is highly expressed during spine formation and progressively declines after synaptic maturation (Moya et al., 1994). Recently APP has been described to affect spine density and to play a key role in learning and memory. Increased APP levels in brain result in increased synaptic density (Lee et al., 2010b), which is also a feature of FMRP genetic ablation (Bagni and Greenough, 2005). In agreement with previous findings from Lee and co-workers in rat neurons (Lee et al., 2010), we found here that overexpression of APP in mouse cortical neurons increases spine number, whereas knockdown of APP reduced spine density in cultured hippocampal neurons (FIG. 5) further consolidating a role of APP in spine formation.

The α-secretase Pathway is Dysregulated in FXS

There is extensive evidence that APP expression is potentially regulated by post-transcriptional mechanisms such as APP mRNA stabilization and APP translation, indicating that the regulation of APP mRNA metabolism is an important event in AD pathophysiology. Furthermore, we have recently shown that App mRNA is associated to the Cytoplasmic FMRP Interacting Protein 1 (CYFIP1) and its protein level appeared increased in the CYFIP1+/−mice (Napoli et al., 2008). Importantly, the CYFIP1 gene has been linked to Autism and sAPP level is increased in autistic patients (Rey 2011). Since the FMRP-CYFIP1 complex controls mRNA translation at synapses and it is regulated by synaptic activity we investigated if App mRNA is translationally controlled by the FMRP-CYFIP complex. We show here, through a canonical polysomes-mRNPs analysis that App mRNA as well the mRNA encoding the a-secretase ADAM10 are translationally regulated by FMRP (FIG. 3). Since FMRP has a major impact on brain mRNA metabolism (Bagni and Greenough 2005, De Rubeis and Bagni 2010, Bassel and Warren 2008), it is of fundamental importance to have identified App and Adam10 mRNAs as amongst the targets dysregulated in FXS. FMRP binds target mRNAs directly through the recognition of specific elements, including G-rich (Dreyfus et al 2001; Adinolfi et al 1999; Zalfa et al 2007) or G-quadruplex (G-quartet) structures (Darnell et al., 2001; Phan et al., 2011; Subramanian et al., 2011) or via non-coding RNAs such as BC1 or microRNAs (Bagni and Greenough 2005, Edbauer et al 2010; Bassell 2011). Interestingly, both App and Adam10 mRNA bear a putative G-quartet sequence embedded into a G-rich region in its 5′UTR (FIG. 13). These regions have been implicated in both translational inhibition (Schaeffer C et al., 2001; Lammich et al., 2010; Morris M J et al., 2010, Shahid R et al., 2010, Gomez D et al., 2010, Abdelmohsen K et al., 2011) and mRNA stability (D'Orso I et al., 2001, Zalfa et al., 2007). Absence of FMRP does not affect App and Adam10 mRNA stability (FIG. 9 and FIG. 4C) but their mRNA translation.

Recently, the three-dimensional structure from the FMRP RGG box domain together with a G quartet-like RNA sequence has been obtained through nuclear magnetic resonance (Phan et al 2011) further supporting the functional interaction between FMRP and G-rich regions. At the translational level, ADAM10 expression is suppressed through mechanisms involving the 5′UTR of Adam10 mRNA (Lammich et al., 2010). Furthermore we show here that translation of App and Adam10 is regulated by FMRP in an activity-dependent manner (FIG. 2 and FIG. 3). Besides the increase of APP, the concomitant increase of ADAM10 in FXS reinforces the importance of the non-amyloidogenic pathway leading to the production of increased APPa (FIGS. 3-4) and its effect rescuing the spine phenotype (FIG. 5).

Recently, postnatal Disruption of the Disintegrin/Metalloproteinase ADAM10 in brain revealed features shared in Fragile X such as epileptic seizures, learning deficits, altered spine morphology, and defective synaptic functions (Prox J et al., 2013). These findings further highlight the required correct level of ADAM10 for correct brain functions.

The molecular mechanisms underlying the competition between a- and b-secretase are not yet fully understood but may involve changes in the cellular compartments, where the cleavage typically takes place. a-secretase cleavage occurs at the plasma membrane, whereas b-secretase cleavage mostly occurs in endosomes (FIG. 7A). A reduction of APP endocytosis increases APP levels at the cell surface, resulting in enhanced APP cleavage by a-secretase and reduced Ab levels (reviewed in Lichtenthaler 2006). Finally, in conditional Adam10 KO mice sAPPa production is nearly completely abolished demonstrating that ADAM10 is required for constitutive sAPPa cleavage in neurons (Jorissen et al., 2010).

Envisioning an APP Modulation as Treatment for FXS

Several in vitro and in vivo evidences show that sAPPα has a physiological function in neurons. In cortical and hippocampal neuronal cells (Araki et al., 1991; Ohsawa et al., 1997; Qiu et al., 1995) and human neuroblastoma cell lines (Wang et al., 2004) sAPPa levels interferes with neurite outgrowth. In vivo application of sAPPα increases synaptic density, memory retention (Roch et al., 1994) and improves performance in tasks involved in short and long term memory and causes proliferation of progenitor cells in the adult subventricular zone (Caille et al., 2004). Since both exogenously infused sAPPa in brain and endogenously overexpressed ADAM10 have neurotrophic effects on cortical synaptogenesis (Bell eta al 2008) and ADAM10 synaptic localization and activity is important for synaptic morphology (Malinverno et al., 2010), we believe that this pathway is strongly contributing to the FXS phenotype. APP dysregulation during a precise developmental window (P21, FIG. 1) strongly support our hypothesis and are in agreement.

We show here that the rescue of spine density in FXS upon sAPPα administration (FIG. 5) occurs in a time window that precedes synaptic consolidation in vitro (DIV 7), highlighting the effect's that APP-ADAM10 combination has on FXS. We propose that the APP-ADAM10 pathway may be a new avenue to intervene in patients with FXS.

Interestingly, one of the metalloproteinase that seem to affect APP processing is the matrix metalloproteinase 9 (MMP-9). MMP9 has been largely associated to cancer progression and metastasis and recent evidence seem to point out to its role in APP processing as well (Mizoguchi H et al., 2009; Yan P et al., 2006; Backstrom et al., 1996). MMP9 has been recently found upregulated in FXS mice (Bilousova et al., 2008) and MMP-9 overexpression causes elongation and thinning of dendritic spines (Michaluk et al 2011). These findings support a possible concerted action of MMP9 and ADAM10 on the increased sAPPs observed in FXS. Importantly minocycline reduces excess of MMP-9 activity. Newborn Fmr1 KO mice when treated with minocycline showed a rescue of the dendritic spine deficits and improvement in anxiety along with more strategic exploratory behavior (Bilousova et al., 2008).

Moreover, the relevance of ADAM10 as therapeutic target for innovative therapy of Fragile X is proved by in vivo experiments. We used the cell permeable peptide TAT-Pro to inhibit ADAM10 trafficking and, thereby, its shedding activity. TAT-Pro treatment was able to reduce sAPPα levels in young Fmr1 KO mice exposed to the peptide for 24 h. Furthermore, the analysis of spine morphology revealed that TAT-Pro administration significantly modified spines length and width, rescuing the pathological phenotype of Fmr1 KO mice. Not only the spines are modified, but mice treated with this inhibitor also show improved behavioural effects.

Since the cell-permeable peptide-treatment turned out to be effective, these outcomes pave the way to a new approach of treating neurodevelopmental disorders by repairing the synaptic damage.

### REFERENCES

Bagni, C., and Greenough, W. T. (2005). From mRNP trafficking to spine dysmorphogenesis: the roots of fragile X syndrome. Nat Rev Neurosci 6, 376-387.
Bassell, G. J., and Warren, S. T. (2008). Fragile X syndrome: loss of local mRNA regulation alters synaptic development and function. Neuron 60, 201-214.
Bittner, T., Fuhrmann, M., Burgold, S., Jung, C. K., Volbracht, C., Steiner, H., Mitteregger, G., Kretzschmar, H. A., Haass, C., and Herms, J. (2009). Gamma-secretase inhibition reduces spine density in vivo via an amyloid precursor protein-dependent pathway. The Journal of neuroscience: the official journal of the Society for Neuroscience 29, 10405-10409.
Cruz-Martin, A., Crespo, M., and Portera-Cailliau, C. (2010). Delayed stabilization of dendritic spines in fragile X mice. The Journal of neuroscience: the official journal of the Society for Neuroscience 30, 7793-7803.
Dolen, G., Osterweil, E., Rao, B. S., Smith, G. B., Auerbach, B. D., Chattarji, S., and Bear, M. F. (2007). Correction of fragile X syndrome in mice. Neuron 56, 955-962.
Ferrari, F., Mercaldo, V., Piccoli, G., Sala, C., Cannata, S., Achsel, T., and Bagni, C. (2007). The fragile X mental retardation protein-RNP granules show an mGluR-dependent localization in the post-synaptic spines. Mol Cell Neurosci 34, 343-354.
Giuffrida, R., Musumeci, S., D'Antoni, S., Bonaccorso, C. M., Giuffrida-Stella, A. M., Oostra, B. A., and Catania, M. V. (2005). A reduced number of metabotropic glutamate subtype 5 receptors are associated with constitutive homer proteins in a mouse model of fragile X syndrome. The Journal of neuroscience : the official journal of the Society for Neuroscience 25, 8908-8916.
Gross, C., Berry-Kravis, E. M., and Bassell, G. J. (2012). Therapeutic Strategies in Fragile X Syndrome: Dysregulated mGluR Signaling and Beyond. Neuropsychopharmacology 37, 178-195.
Hartmann, D., de Strooper, B., Serneels, L., Craessaerts, K., Herreman, A., Annaert, W., Umans, L., Lubke, T., Lena Illert, A., von Figura, K., et al. (2002). The disintegrin/metalloprotease ADAM 10 is essential for Notch signalling but not for alpha-secretase activity in fibroblasts. Human molecular genetics 11, 2615-2624.
Hoe, H. S., Fu, Z., Makarova, A., Lee, J. Y., Lu, C., Feng, L., Pajoohesh-Ganji, A., Matsuoka, Y., Hyman, B. T., Ehlers, M. D., et al. (2009). The effects of amyloid precursor protein on postsynaptic composition and activity. The Journal of biological chemistry 284, 8495-8506.
Hoettecke N, Ludwig A, Foro S, Schmidt B. Improved synthesis of ADAM10 inhibitor G1254023X. Neurodegener Dis. 2010; 7(4):232-8.
Hou, L., Antion, M. D., Hu, D., Spencer, C. M., Paylor, R., and Klann, E. (2006). Dynamic translational and proteasomal regulation of fragile X mental retardation protein controls mGluR-dependent long-term depression. Neuron 51, 441-454.
Huber, K. M., Gallagher, S. M., Warren, S. T., and Bear, M. F. (2002). Altered synaptic plasticity in a mouse model of fragile X mental retardation. Proceedings of the National Academy of Sciences of the United States of America 99, 7746-7750.
Hurst L A, Bunning R A, Sharrack B, Woodroofe M N. siRNA knockdown of ADAM-10, but not ADAM-17, significantly reduces fractalkine shedding following pro-inflammatory cytokine treatment in a human adult brain endothelial cell line. Neurosci Lett. 2012; 521(1):52-6.
Jorissen, E., Prox, J., Bernreuther, C., Weber, S., Schwanbeck, R., Serneels, L., Snellinx, A., Craessaerts, K., Thathiah, A., Tesseur, I., et al. (2010). The disintegrin/metalloproteinase ADAM10 is essential for the establishment of the brain cortex. The Journal of neuroscience: the official journal of the Society for Neuroscience 30, 4833-4844.
Kins, S., Lauther, N., Szodorai, A., and Beyreuther, K. (2006). Subcellular trafficking of the amyloid precursor protein gene family and its pathogenic role in Alzheimer's disease. Neuro-degenerative diseases 3, 218-226.
Kuhn, P. H., Wang, H., Dislich, B., Colombo, A., Zeitschel, U., Ellwart, J. W., Kremmer, E., Rossner, S., and Lichtenthaler, S. F. (2010). ADAM10 is the physiologically relevant, constitutive alpha-secretase of the amyloid precursor protein in primary neurons. EMBO J 29, 3020-3032.
Lammich, S., Buell, D., Zilow, S., Ludwig, A. K., Nuscher, B., Lichtenthaler, S. F., Prinzen, C., Fahrenholz, F., and Haass, C. (2010). Expression of the anti-amyloidogenic secretase ADAM10 is suppressed by its 5′-untranslated region. The Journal of biological chemistry 285, 15753-15760.
Lammich, S., Kamp, F., Wagner, J., Nuscher, B., Zilow, S., Ludwig, A. K., Willem, M., and Haass, C. (2011). Translational Repression of the Disintegrin and Metalloprotease ADAM10 by a Stable G-quadruplex Secondary Structure in Its 5′-Untranslated Region. The Journal of biological chemistry 286, 45063-45072.
Lammich, S., Kojro, E., Postina, R., Gilbert, S., Pfeiffer, R., Jasionowski, M., Haass, C., and Fahrenholz, F. (1999). Constitutive and regulated alpha-secretase cleavage of Alzheimer's amyloid precursor protein by a disintegrin metalloprotease. Proceedings of the National Academy of Sciences of the United States of America 96, 3922-3927.
Lee, E. K., Kim, H. H., Kuwano, Y., Abdelmohsen, K., Srikantan, S., Subaran, S. S., Gleichmann, M., Mughal, M. R., Martindale, J. L., Yang, X., et al. (2010a). hnRNP C promotes APP translation by competing with FMRP for APP mRNA recruitment to P bodies. Nat Struct Mol Biol 17, 732-739.
Lee, K. J., Moussa, C. E., Lee, Y., Sung, Y., Howell, B. W., Turner, R. S., Pak, D. T., and Hoe, H. S. (2010b). Beta amyloid-independent role of amyloid precursor protein in generation and maintenance of dendritic spines. Neuroscience 169, 344-356.
Li, J., Pelletier, M. R., Perez Velazquez, J. L., and Carlen, P. L. (2002). Reduced cortical synaptic plasticity and GIuR1 expression associated with fragile X mental retardation protein deficiency. Mol Cell Neurosci 19, 138-151.
Lichtenthaler, S. F. (2011). Alpha-secretase in Alzheimer's disease: molecular identity, regulation and therapeutic potential. Journal of neurochemistry 116, 10-21.
Liu-Yesucevitz, L., Bassell, G. J., Gitler, A. D., Hart, A. C., Klann, E., Richter, J. D., Warren, S. T., and Wolozin, B. (2011). Local RNA translation at the synapse and in disease. The Journal of neuroscience : the official journal of the Society for Neuroscience 31, 16086-16093.
Lu, R., Wang, H., Liang, Z., Ku, L., O'Donnell W, T., Li, W., Warren, S. T., and Feng, Y. (2004). The fragile X protein controls microtubule-associated protein 1B translation and microtubule stability in brain neuron development. Proceedings of the National Academy of Sciences of the United States of America 101, 15201-15206.
Malinverno, M., Carta, M., Epis, R., Marcello, E., Verpelli, C., Cattabeni, F., Sala, C., Mulle, C., Di Luca, M., and Gardoni, F. (2010). Synaptic localization and activity of ADAM10 regulate excitatory synapses through N-cadherin cleavage. The Journal of neuroscience: the official journal of the Society for Neuroscience 30, 16343-16355.
Moya, K. L., Benowitz, L. I., Schneider, G. E., and Allinquant, B. (1994). The amyloid precursor protein is developmentally regulated and correlated with synaptogenesis. Dev Biol 161, 597-603.
Napoli, I., Mercaldo, V., Boyl, P. P., Eleuteri, B., Zalfa, F., De Rubeis, S., Di Marino, D., Mohr, E., Massimi, M., Falconi, M., et al. (2008). The fragile X syndrome protein represses activity-dependent translation through CYFIP1, a new 4E-BP. Cell 134, 1042-1054.
Pfeiffer, B. E., and Huber, K. M. (2009). The state of synapses in fragile X syndrome. Neuroscientist 15, 549-567.
Soundararajan R, Sayat R, Robertson G S, Marignani P A. Triptolide: An inhibitor of a disintegrin and metalloproteinase 10 (ADAM10) in cancer cells. Cancer Biol Ther. 2009; 8(21):2054-62.
Tomai E, Butz K, Lohrey C, von Weizsacker F, Zentgraf H, Hoppe-Seyler F. Peptide aptamer-mediated inhibition of target proteins by sequestration into aggresomes. J Biol Chem. 2006; 281(30):21345-52.
Torroja, L., Packard, M., Gorczyca, M., White, K., and Budnik, V. (1999). The Drosophila beta-amyloid precursor protein homolog promotes synapse differentiation at the neuromuscular junction. The Journal of neuroscience: the official journal of the Society for Neuroscience 19, 7793-7803.
Vingtdeux, V., and Marambaud, P. (2012). Identification and biology of alpha-secretase. Journal of neurochemistry 120 Suppl 1, 34-45.
Wang, Z., Wang, B., Yang, L., Guo, Q., Aithmitti, N., Songyang, Z., and Zheng, H. (2009). Presynaptic and postsynaptic interaction of the amyloid precursor protein promotes peripheral and central synaptogenesis. The Journal of neuroscience: the official journal of the Society for Neuroscience 29, 10788-10801.
Weiler, I. J., Irwin, S. A., Klintsova, A. Y., Spencer, C. M., Brazelton, A. D., Miyashiro, K., Comery, T. A., Patel, B., Eberwine, J., and Greenough, W. T. (1997). Fragile X mental retardation protein is translated near synapses in response to neurotransmitter activation. Proceedings of the National Academy of Sciences of the United States of America 94, 5395-5400.
Westmark, C. J., and Malter, J. S. (2007). FMRP mediates mGluR5-dependent translation of amyloid precursor protein. PLoS Biol 5, e52.
Zalfa, F., Eleuteri, B., Dickson, K. S., Mercaldo, V., De Rubeis, S., di Penta, A., Tabolacci, E., Chiurazzi, P., Neri, G., Grant, S. G., et al. (2007). A new function for the fragile X mental retardation protein in regulation of PSD-95 mRNA stability. Nat Neurosci 10, 578-587.
Zalfa, F., Giorgi, M., Primerano, B., Moro, A., Di Penta, A., Reis, S., Oostra, B., and Bagni, C. (2003). The fragile X syndrome protein FMRP associates with BC1 RNA and regulates the translation of specific mRNAs at synapses. Cell 112, 317-327.
Zhang, Y. W., Thompson, R., Zhang, H., and Xu, H. (2011). APP processing in Alzheimer's disease. Mol Brain 4, 3.
Zheng, H., and Koo, E. H. (2011). Biology and pathophysiology of the amyloid precursor protein. Mol Neurodegener 6, 27.
Zhou, L., Brouwers, N., Benilova, I., Vandersteen, A., Mercken, M., Van Laere, K., Van Damme, P., Demedts, D., Van Leuven, F., Sleegers, K., et al. (2011). Amyloid precursor protein mutation E682K at the alternative beta-secretase cleavage beta′-site increases Abeta generation. EMBO Mol Med 3, 291-302.

1. An inhibitor of ADAM10 for use in treatment of Fragile X syndrome.

2. The inhibitor according to claim 1, selected from an anti-ADAM10 peptide, GI254023X and triptolide.

3. The inhibitor according to claim 2, wherein the anti-ADAM10 peptide contains the sequence YGRKKRRQRRRPKLPPPKPLPGTLKRRRPPQP.

4. The inhibitor according to claim 3, wherein the anti-ADAM10 peptide is the Tat-Pro ADAM ¹⁰⁰⁹⁻⁷²⁹ peptide.

5. The inhibitor according to claim 1, which rescues spine dysmorphogenesis.
